SEQUENCE LISTING (6 sequences)

SEQ ID NO: 1 - DNA, 8686 bp, Homo sapiens
  CDS (157)..(7677)
gagtctttgaggacacagcctcgctggaggcagtttctggtgccagtgacggggtggcccgtgagctgatgacgaggactggcttttaatccttggtggtgattaagagaaagcttattggggcctgggagcagctccccgccgacccccaccaccatgtcgggctccacacagcctgtggcacagacgtggagggccactgagccccgctacccgccccacagcctttcctacccagtgcagatcgcccggacgcacacggacgtcgggctcctggagtaccagcaccactcccgcgactatgcctcccacctgtcgcccggctccatcatccagccccagcggcggaggccctccctgctgtctgagttccagcccgggaatgaacggtcccaggagctccacctgcggccagagtcccactcatacctgcccgagctggggaagtcagagatggagttcattgaaagcaagcgccctcggctagagctgctgcctgaccccctgctgcgaccgtcacccctgctggccacgggccagcctgcgggatctgaagacctcaccaaggaccgtagcctgacgggcaagctggaaccggtgtctccccccagccccccgcacactgaccctgagctggagctggtgccgccacggctgtccaaggaggagctgatccagaacatggaccgcgtggaccgagagatcaccatggtagagcagcagatctctaagctgaagaagaagcagcaacagctggaggaggaggctgccaagccgcccgagcctgagaagcccgtgtcaccgccgcccatcgagtcgaagcaccgcagcctggtgcagatcatctacgacgagaaccggaagaaggctgaagctgcacatcggattctggaaggcctggggccccaggtggagctgccgctgtacaaccagccctccgacacccggcagtatcatgagaacatcaaaataaaccaggcgatgcggaagaagctaatcttgtacttcaagaggaggaatcacgctcggaaacaatgggagcagaagttctgccagcgctatgaccagctcatggaggcctgggagaagaaggtggagcgcatcgagaacaacccccggcggcgggccaaggagagcaaggttcgcgagtactacgagaagcagttccctgagatccgcaagcagcgcgagctgcaggagcgcatgcagagggtgggccagcggggcagtgggctgtccatgtcgcccgcccgcagcgagcacgaggtgtcagagatcatcgatggcctctcagagcaggagaacctggagaagcagatgcgccagctggccgtgatcccgcccatgctgtacgacgctgaccagcagcgcatcaagttcatcaacatgaacgggcttatggccgaccccatgaaggtgtacaaagaccgccaggtcatgaacatgtggagtgagcaggagaaggagaccttccgggagaagttcatgcagcatcccaagaactttggcctgatcgcatcattcctggagaggaagacagtggctgagtgcgtcctctattactacctgactaagaagaatgagaactataagagcctggtgagacggagctatcggcgccgcggcaagagccagcagcaacaacagcagcagcagcagcagcagcagcagcagcagcagcagcccatgccccgcagcagccaggaggagaaagatgagaaggagaaggaaaaggaggcggagaaggaggaggagaagccggaggtggagaacgacaaggaagacctcctcaaggagaagacagacgacacctcaggggaggacaacgacgagaaggaggctgtggcctccaaaggccgcaaaactgccaacagccagggaagacgcaaaggccgcatcacccgctcaatggctaatgaggccaacagcgaggaggccatcaccccccagcagagcgccgagctggcctccatggagctgaatgagagttctcgctggacagaagaagaaatggaaacagccaagaaaggtctcctggaacacggccgcaactggtcggccatcgcccggatggtgggctccaagactgtgtcgcagtgtaagaacttctacttcaactacaagaagaggcagaacctcgatgagatcttgcagcagcacaagctgaagatggagaaggagaggaacgcgcggaggaagaagaagaaagcgccggcggcggccagcgaggaggctgcattcccgcccgtggtggaggatgaggagatggaggcgtcgggcgtgacgggaaatgaggaggagatggtggaggaggctgaagccactgtcaacaacagctcagacaccgagagcatcccctctcctcacactgaggccgccaaggacacagggcagaatgggcccaagcccccagccaccctgggcgccgacgggccacccccagggccacccaccccaccaccggaggacatcccggcccccactgagtccaccccggcctctgaagccaccttagcccctacgcccccaccagcacccccatttccctcttcacctcctcctgtggtccccaaggaggagaaggaggaggagaccgcagcagcgcccccagtggaggagggggaggagcagaagccccccgcggctgaggagctggcagtggacacagggaaggccgaggagcccgtcaagagcgagtgcacggaggaagccgaggaggggccggccaagggcaaggacgcggaggccgctgaggccacggccgagagggcgctcaaggcagagaagaaggagggcgggagcggcagggccaccacagccaagagctcgggcgccccccaggacagcgactccagtgccacctgcagtgcagacgaggtggatgaggccgagggcggcgacaagaaccggctgctgtccccaaggcccagcctcctcaccccgactggcgacccccgggccaatgcctcaccccagaagccactggacctgaagcagctgaagcagcgagcggctgccatcccccccatccaggtcaccaaagtccatgagcccccccgggaggacgcagctcccaccaagccagctcccccagccccaccgccaccgcaaaacctgcagccggagagcgacgcccctcagcagcctggcagcagcccccggggcaagagcaggagcccggcaccccccgccgacaaggaggcagagaagcctgtgttcttcccagccttcgcagccgaggcccagaagctgcctggggaccccccttgctggacttccggcctgcccttccccgtgcccccccgtgaggtgatcaaggcctccccgcatgccccggacccctcagccttctcctacgctccacctggtcacccactgcccctgggcctccatgacactgcccggcccgtcctgccgcgcccacccaccatctccaacccgcctcccctcatctcctctgccaagcaccccagcgtcctcgagaggcaaataggtgccatctcccaaggaatgtcggtccagctccacgtcccgtactcagagcatgccaaggccccggtgggccctgtcaccatggggctgcccctgcccatggaccccaaaaagctggcacccttcagcggagtgaagcaggagcagctgtccccacggggccaggctgggccaccggagagcctgggggtgcccacagcccaggaggcgtccgtgctgagagggacagctctgggctcagttccgggcggaagcatcaccaaaggcattcccagcacacgggtgccctcggacagcgccatcacataccgcggctccatcacccacggcacgccagctgacgtcctgtacaagggcaccatcaccaggatcatcggcgaggacagcccgagtcgcttggaccgcggccgggaggacagcctgcccaagggccacgtcatctacgaaggcaagaagggccacgtcttgtcctatgagggtggcatgtctgtgacccagtgctccaaggaggacggcagaagcagctcaggacccccccatgagacggccgcccccaagcgcacctatgacatgatggagggccgcgtgggcagagccatctcctcagccagcatcgaaggtctcatgggccgtgccatcccgccggagcgacacagcccccaccacctcaaagagcagcaccacatccgcgggtccatcacacaagggatccctcggtcctacgtggaggcacaggaggactacctgcgtcgggaggccaagctcctaaagcgggagggcacgcctccgcccccaccgccctcacgggacctgaccgaggcctacaagacgcaggccctgggccccctgaagctgaagccggcccatgagggcctggtggccacggtgaaggaggcgggccgctccatccatgagatcccgcgcgaggagctgcggcacacgcccgagctgcccctggccccgcggccgctcaaggagggctccatcacgcagggcaccccgctcaagtacgacaccggcgcgtccaccactggctccaaaaagcacgacgtacgctccctcatcggcagccccggccggacgttcccacccgtgcacccgctggatgtgatggccgacgcccgggcactggaacgtgcctgctacgaggagagcctgaagagccggccagggaccgccagcagctcggggggctccattgcgcgcggcgccccggtcattgtgcctgagctgggtaagccgcggcagagccccctgacctatgaggaccacggggcaccctttgccggccacctcccacgaggttcgcccgtgaccatgcgggagcccacgccgcgcctgcaggagggcagcctttcgtccagcaaggcatcccaggaccgaaagctgacgtcgacgcctcgtgagatcgccaagtccccgcacagcaccgtgcccgagcaccacccacaccccatctcgccctatgagcacctgcttcggggcgtgagtggcgtggacctgtatcgcagccacatccccctggccttcgaccccacctccataccccgcggcatccctctggacgcagccgctgcctactacctgccccgacacctggcccccaaccccacctacccgcacctgtacccaccctacctcatccgcggctaccccgacacggcggcgctggagaaccggcagaccatcatcaatgactacatcacctcgcagcagatgcaccacaacacggccaccgccatggcccagcgagctgatatgctgaggggcctctcgccccgcgagtcctcgctggcactcaactacgctgcgggtccccgaggcatcatcgacctgtcccaagtgccacacctgcctgtgctcgtgcccccgacaccaggcaccccagccaccgccatggaccgccttgcctacctccccaccgcgccccagcccttcagcagccgccacagcagctccccactctccccaggaggtccaacacacttgacaaaaccaaccaccacgtcctcgtccgagcgggagcgagaccgggatcgagagcgggaccgggatcgggagcgggaaaagtccatcctcacgtccaccacgacggtggagcacgcacccatctggagacctggtacagagcagagcagcggcagcagcggcagcagcggcgggggtgggggcagcagcagccgccccgcctcccactcccatgcccaccagcactcgcccatctcccctcggacccaggatgccctccagcagagacccagtgtgcttcacaacacaggcatgaagggtatcatcaccgctgtggagcccagcaagcccacggtcctgaggtccacctccacctcctcacccgttcgcccagctgccacattcccacctgccacccactgcccactgggcggcaccctcgatggggtctaccctaccctcatggagcccgtcttgctgcccaaggaggccccccgggtcgcccggccagagcggccccgagcagacaccggccatgccttcctcgccaagcccccagcccgctccgggctggagcccgcctcctcccccagcaagggctcggagccccggcccctagtgcctcctgtctctggccacgccaccatcgcccgcacccctgcgaagaacctcgcacctcaccacgccagcccggacccgccggcgccacctgcctcggcctcggacccgcaccgggaaaagactcaaagtaaacccttttccatccaggaactggaactccgttctctgggttaccacggcagcagctacagccccgaaggggtggagcccgtcagccctgtgagctcacccagtctgacccacgacaaggggctccccaagcacctggaagagctcgacaagagccacctggagggggagctgcggcccaagcagccaggccccgtgaagcttggcggggaggccgcccacctcccacacctgcggccgctgcctgagagccagccctcgtccagcccgctgctccagaccgccccaggggtcaaaggtcaccagcgggtggtcaccctggcccagcacatcagtgaggtcatcacacaggactacacccggcaccacccacagcagctcagcgcacccctgcccgcccccctctactccttccctggggccagctgccccgtcctggacctccgccgcccacccagtgacctctacctcccgcccccggaccatggtgccccggcccgtggctccccccacagcgaagggggcaagaggtctccagagccaaacaagacgtcggtcttgggtggtggtgaggacggtattgaacctgtgtccccaccggagggcatgacggagccagggcactcccggagtgctgtgtacccgctgctgtaccgggatggggaacagacggagcccagcaggatgggctccaagtctccaggcaacaccagccagccgccagccttcttcagcaagctgaccgagagcaactccgccatggtcaagtccaagaagcaagagatcaacaagaagctgaacacccacaaccggaatgagcctgaatacaatatcagccagcctgggacggagatcttcaatatgcccgccatcaccggaacaggccttatgacctatagaagccaggcggtgcaggaacatgccagcaccaacatggggctggaggccataattagaaaggcactcatgggtaaatatgaccagtgggaagagtccccgccgctcagcgccaatgcttttaaccctctgaatgccagtgccagcctgcccgctgctatgcccataaccgctgctgacggacggagtgaccacacactcacctcgccaggtggcggcgggaaggccaaggtctctggcagacccagcagccgaaaagccaagtccccggccccgggcctggcatctggggaccggccaccctctgtctcctcagtgcactcggagggagactgcaaccgccggacgccgctcaccaaccgcgtgtgggaggacaggccctcgtccgcaggttccacgccattcccctacaaccccctgatcatgcggctgcaggcgggtgtcatggcttccccacccccaccgggcctccccgcgggcagcgggcccctcgctggcccccaccacgcctgggacgaggagcccaagccactgctctgctcgcagtacgagacactctccgacagcgagtgactcagaacagggcgggggggggcgggcggtgtcaggtcccagcgagccacaggaacggccctgcaggagcggggcggctgccgactcccccaaccaaggaaggagcccctgagtccgcctgcgcctccatccatctgtccgtccagagccggcatccttgcctgtctaaagccttaactaagactcccgccccgggctggccctgtgcagaccttactcaggggatgtttacctggtgctcgggaagggaggggaaggggccggggagggggcacggcaggcgtgtggcagccacacacaggcggccagggcggccagggacccaaagcaggatgaccacgcacctccacgccactgcctcccccgaatgcatttggaaccaaagtctaaactgagctcgcagcccccgcgccctccctccgcctcccatcccgcttagcgctctggacagatggacgcaggccctgtccagcccccagtgcgctcgttccggtccccacagactgccccagccaacgagattgctggaaaccaagtcaggccaggtgggcggacaaaagggccaggtgcggcctggggggaacggatgctccgaggactggactgtttttttcacacatcgttgccgcagcggtgggaaggaaaggcagatgtaaatgatgtgttggtttacagggtatatttttgataccttcaatgaattaattcagatgttttacgcaaggaaggacttacccagtattactgctgctgtgcttttgatctctgcttaccgttcaagaggcgtgtgcaggccgacagtcggtgaccccatcactcgcaggaccaagggggcggggactgctcgtcacgccccgctgtgtcctccctccctcccttccttgggcagaatgaattcgatgcgtattctgtggccgccatttgcgcagggtggtggtattctgtcatttacacacgtcgttctaattaaaaagcgaattatactccaaaaaaaaaaaaaaaaaaa

SEQ ID NO: 2 - protein, 2507 aa, Homo sapiens
MSGSTQPVAQTWRATEPRYPPHSLSYPVQIARTHTDVGLLEYQHHSRDYASHLSPGSIIQPQRRRPSLLSEFQPGNERSQELHLRPESHSYLPELGKSEMEFIESKRPRLELLPDPLLRPSPLLATGQPAGSEDLTKDRSLTGKLEPVSPPSPPHTDPELELVPPRLSKEELIQNMDRVDREITMVEQQISKLKKKQQQLEEEAAKPPEPEKPVSPPPIESKHRSLVQIIYDENRKKAEAAHRILEGLGPQVELPLYNQPSDTRQYHENIKINQAMRKKLILYFKRRNHARKQWEQKFCQRYDQLMEAWEKKVERIENNPRRRAKESKVREYYEKQFPEIRKQRELQERMQRVGQRGSGLSMSPARSEHEVSEIIDGLSEQENLEKQMRQLAVIPPMLYDADQQRIKFINMNGLMADPMKVYKDRQVMNMWSEQEKETFREKFMQHPKNFGLIASFLERKTVAECVLYYYLTKKNENYKSLVRRSYRRRGKSQQQQQQQQQQQQQQQQQPMPRSSQEEKDEKEKEKEAEKEEEKPEVENDKEDLLKEKTDDTSGEDNDEKEAVASKGRKTANSQGRRKGRITRSMANEANSEEAITPQQSAELASMELNESSRWTEEEMETAKKGLLEHGRNWSAIARMVGSKTVSQCKNFYFNYKKRQNLDEILQQHKLKMEKERNARRKKKKAPAAASEEAAFPPVVEDEEMEASGVTGNEEEMVEEAEATVNNSSDTESIPSPHTEAAKDTGQNGPKPPATLGADGPPPGPPTPPPEDIPAPTESTPASEATLAPTPPPAPPFPSSPPPVVPKEEKEEETAAAPPVEEGEEQKPPAAEELAVDTGKAEEPVKSECTEEAEEGPAKGKDAEAAEATAERALKAEKKEGGSGRATTAKSSGAPQDSDSSATCSADEVDEAEGGDKNRLLSPRPSLLTPTGDPRANASPQKPLDLKQLKQRAAAIPPIQVTKVHEPPREDAAPTKPAPPAPPPPQNLQPESDAPQQPGSSPRGKSRSPAPPADKEAEKPVFFPAFAAEAQKLPGDPPCWTSGLPFPVPPREVIKASPHAPDPSAFSYAPPGHPLPLGLHDTARPVLPRPPTISNPPPLISSAKHPSVLERQIGAISQGMSVQLHVPYSEHAKAPVGPVTMGLPLPMDPKKLAPFSGVKQEQLSPRGQAGPPESLGVPTAQEASVLRGTALGSVPGGSITKGIPSTRVPSDSAITYRGSITHGTPADVLYKGTITRIIGEDSPSRLDRGREDSLPKGHVIYEGKKGHVLSYEGGMSVTQCSKEDGRSSSGPPHETAAPKRTYDMMEGRVGRAISSASIEGLMGRAIPPERHSPHHLKEQHHIRGSITQGIPRSYVEAQEDYLRREAKLLKREGTPPPPPPSRDLTEAYKTQALGPLKLKPAHEGLVATVKEAGRSIHEIPREELRHTPELPLAPRPLKEGSITQGTPLKYDTGASTTGSKKHDVRSLIGSPGRTFPPVHPLDVMADARALERACYEESLKSRPGTASSSGGSIARGAPVIVPELGKPRQSPLTYEDHGAPFAGHLPRGSPVTMREPTPRLQEGSLSSSKASQDRKLTSTPREIAKSPHSTVPEHHPHPISPYEHLLRGVSGVDLYRSHIPLAFDPTSIPRGIPLDAAAAYYLPRHLAPNPTYPHLYPPYLIRGYPDTAALENRQTIINDYITSQQMHHNTATAMAQRADMLRGLSPRESSLALNYAAGPRGIIDLSQVPHLPVLVPPTPGTPATAMDRLAYLPTAPQPFSSRHSSSPLSPGGPTHLTKPTTTSSSERERDRDRERDRDREREKSILTSTTTVEHAPIWRPGTEQSSGSSGSSGGGGGSSSRPASHSHAHQHSPISPRTQDALQQRPSVLHNTGMKGIITAVEPSKPTVLRSTSTSSPVRPAATFPPATHCPLGGTLDGVYPTLMEPVLLPKEAPRVARPERPRADTGHAFLAKPPARSGLEPASSPSKGSEPRPLVPPVSGHATIARTPAKNLAPHHASPDPPAPPASASDPHREKTQSKPFSIQELELRSLGYHGSSYSPEGVEPVSPVSSPSLTHDKGLPKHLEELDKSHLEGELRPKQPGPVKLGGEAAHLPHLRPLPESQPSSSPLLQTAPGVKGHQRVVTLAQHISEVITQDYTRHHPQQLSAPLPAPLYSFPGASCPVLDLRRPPSDLYLPPPDHGAPARGSPHSEGGKRSPEPNKTSVLGGGEDGIEPVSPPEGMTEPGHSRSAVYPLLYRDGEQTEPSRMGSKSPGNTSQPPAFFSKLTESNSAMVKSKKQEINKKLNTHNRNEPEYNISQPGTEIFNMPAITGTGLMTYRSQAVQEHASTNMGLEAIIRKALMGKYDQWEESPPLSANAFNPLNASASLPAAMPITAADGRSDHTLTSPGGGGKAKVSGRPSSRKAKSPAPGLASGDRPPSVSSVHSEGDCNRRTPLTNRVWEDRPSSAGSTPFPYNPLIMRLQAGVMASPPPPGLPAGSGPLAGPHHAWDEEPKPLLCSQYETLSDSE

SEQ ID NO: 3 - DNA, 7521 bp, Homo sapiens
  CDS (1)..(7521)
atgtcgggctccacacagcctgtggcacagacgtggagggccactgagccccgctacccgccccacagcctttcctacccagtgcagatcgcccggacgcacacggacgtcgggctcctggagtaccagcaccactcccgcgactatgcctcccacctgtcgcccggctccatcatccagccccagcggcggaggccctccctgctgtctgagttccagcccgggaatgaacggtcccaggagctccacctgcggccagagtcccactcatacctgcccgagctggggaagtcagagatggagttcattgaaagcaagcgccctcggctagagctgctgcctgaccccctgctgcgaccgtcacccctgctggccacgggccagcctgcgggatctgaagacctcaccaaggaccgtagcctgacgggcaagctggaaccggtgtctccccccagccccccgcacactgaccctgagctggagctggtgccgccacggctgtccaaggaggagctgatccagaacatggaccgcgtggaccgagagatcaccatggtagagcagcagatctctaagctgaagaagaagcagcaacagctggaggaggaggctgccaagccgcccgagcctgagaagcccgtgtcaccgccgcccatcgagtcgaagcaccgcagcctggtgcagatcatctacgacgagaaccggaagaaggctgaagctgcacatcggattctggaaggcctggggccccaggtggagctgccgctgtacaaccagccctccgacacccggcagtatcatgagaacatcaaaataaaccaggcgatgcggaagaagctaatcttgtacttcaagaggaggaatcacgctcggaaacaatgggagcagaagttctgccagcgctatgaccagctcatggaggcctgggagaagaaggtggagcgcatcgagaacaacccccggcggcgggccaaggagagcaaggttcgcgagtactacgagaagcagttccctgagatccgcaagcagcgcgagctgcaggagcgcatgcagagggtgggccagcggggcagtgggctgtccatgtcgcccgcccgcagcgagcacgaggtgtcagagatcatcgatggcctctcagagcaggagaacctggagaagcagatgcgccagctggccgtgatcccgcccatgctgtacgacgctgaccagcagcgcatcaagttcatcaacatgaacgggcttatggccgaccccatgaaggtgtacaaagaccgccaggtcatgaacatgtggagtgagcaggagaaggagaccttccgggagaagttcatgcagcatcccaagaactttggcctgatcgcatcattcctggagaggaagacagtggctgagtgcgtcctctattactacctgactaagaagaatgagaactataagagcctggtgagacggagctatcggcgccgcggcaagagccagcagcaacaacagcagcagcagcagcagcagcagcagcagcagcagcagcccatgccccgcagcagccaggaggagaaagatgagaaggagaaggaaaaggaggcggagaaggaggaggagaagccggaggtggagaacgacaaggaagacctcctcaaggagaagacagacgacacctcaggggaggacaacgacgagaaggaggctgtggcctccaaaggccgcaaaactgccaacagccagggaagacgcaaaggccgcatcacccgctcaatggctaatgaggccaacagcgaggaggccatcaccccccagcagagcgccgagctggcctccatggagctgaatgagagttctcgctggacagaagaagaaatggaaacagccaagaaaggtctcctggaacacggccgcaactggtcggccatcgcccggatggtgggctccaagactgtgtcgcagtgtaagaacttctacttcaactacaagaagaggcagaacctcgatgagatcttgcagcagcacaagctgaagatggagaaggagaggaacgcgcggaggaagaagaagaaagcgccggcggcggccagcgaggaggctgcattcccgcccgtggtggaggatgaggagatggaggcgtcgggcgtgacgggaaatgaggaggagatggtggaggaggctgaagccactgtcaacaacagctcagacaccgagagcatcccctctcctcacactgaggccgccaaggacacagggcagaatgggcccaagcccccagccaccctgggcgccgacgggccacccccagggccacccaccccaccaccggaggacatcccggcccccactgagtccaccccggcctctgaagccaccttagcccctacgcccccaccagcacccccatttccctcttcacctcctcctgtggtccccaaggaggagaaggaggaggagaccgcagcagcgcccccagtggaggagggggaggagcagaagccccccgcggctgaggagctggcagtggacacagggaaggccgaggagcccgtcaagagcgagtgcacggaggaagccgaggaggggccggccaagggcaaggacgcggaggccgctgaggccacggccgagagggcgctcaaggcagagaagaaggagggcgggagcggcagggccaccacagccaagagctcgggcgccccccaggacagcgactccagtgccacctgcagtgcagacgaggtggatgaggccgagggcggcgacaagaaccggctgctgtccccaaggcccagcctcctcaccccgactggcgacccccgggccaatgcctcaccccagaagccactggacctgaagcagctgaagcagcgagcggctgccatcccccccatccaggtcaccaaagtccatgagcccccccgggaggacgcagctcccaccaagccagctcccccagccccaccgccaccgcaaaacctgcagccggagagcgacgcccctcagcagcctggcagcagcccccggggcaagagcaggagcccggcaccccccgccgacaaggaggcagagaagcctgtgttcttcccagccttcgcagccgaggcccagaagctgcctggggaccccccttgctggacttccggcctgcccttccccgtgcccccccgtgaggtgatcaaggcctccccgcatgccccggacccctcagccttctcctacgctccacctggtcacccactgcccctgggcctccatgacactgcccggcccgtcctgccgcgcccacccaccatctccaacccgcctcccctcatctcctctgccaagcaccccagcgtcctcgagaggcaaataggtgccatctcccaaggaatgtcggtccagctccacgtcccgtactcagagcatgccaaggccccggtgggccctgtcaccatggggctgcccctgcccatggaccccaaaaagctggcacccttcagcggagtgaagcaggagcagctgtccccacggggccaggctgggccaccggagagcctgggggtgcccacagcccaggaggcgtccgtgctgagagggacagctctgggctcagttccgggcggaagcatcaccaaaggcattcccagcacacgggtgccctcggacagcgccatcacataccgcggctccatcacccacggcacgccagctgacgtcctgtacaagggcaccatcaccaggatcatcggcgaggacagcccgagtcgcttggaccgcggccgggaggacagcctgcccaagggccacgtcatctacgaaggcaagaagggccacgtcttgtcctatgagggtggcatgtctgtgacccagtgctccaaggaggacggcagaagcagctcaggacccccccatgagacggccgcccccaagcgcacctatgacatgatggagggccgcgtgggcagagccatctcctcagccagcatcgaaggtctcatgggccgtgccatcccgccggagcgacacagcccccaccacctcaaagagcagcaccacatccgcgggtccatcacacaagggatccctcggtcctacgtggaggcacaggaggactacctgcgtcgggaggccaagctcctaaagcgggagggcacgcctccgcccccaccgccctcacgggacctgaccgaggcctacaagacgcaggccctgggccccctgaagctgaagccggcccatgagggcctggtggccacggtgaaggaggcgggccgctccatccatgagatcccgcgcgaggagctgcggcacacgcccgagctgcccctggccccgcggccgctcaaggagggctccatcacgcagggcaccccgctcaagtacgacaccggcgcgtccaccactggctccaaaaagcacgacgtacgctccctcatcggcagccccggccggacgttcccacccgtgcacccgctggatgtgatggccgacgcccgggcactggaacgtgcctgctacgaggagagcctgaagagccggccagggaccgccagcagctcggggggctccattgcgcgcggcgccccggtcattgtgcctgagctgggtaagccgcggcagagccccctgacctatgaggaccacggggcaccctttgccggccacctcccacgaggttcgcccgtgaccatgcgggagcccacgccgcgcctgcaggagggcagcctttcgtccagcaaggcatcccaggaccgaaagctgacgtcgacgcctcgtgagatcgccaagtccccgcacagcaccgtgcccgagcaccacccacaccccatctcgccctatgagcacctgcttcggggcgtgagtggcgtggacctgtatcgcagccacatccccctggccttcgaccccacctccataccccgcggcatccctctggacgcagccgctgcctactacctgccccgacacctggcccccaaccccacctacccgcacctgtacccaccctacctcatccgcggctaccccgacacggcggcgctggagaaccggcagaccatcatcaatgactacatcacctcgcagcagatgcaccacaacacggccaccgccatggcccagcgagctgatatgctgaggggcctctcgccccgcgagtcctcgctggcactcaactacgctgcgggtccccgaggcatcatcgacctgtcccaagtgccacacctgcctgtgctcgtgcccccgacaccaggcaccccagccaccgccatggaccgccttgcctacctccccaccgcgccccagcccttcagcagccgccacagcagctccccactctccccaggaggtccaacacacttgacaaaaccaaccaccacgtcctcgtccgagcgggagcgagaccgggatcgagagcgggaccgggatcgggagcgggaaaagtccatcctcacgtccaccacgacggtggagcacgcacccatctggagacctggtacagagcagagcagcggcagcagcggcagcagcggcgggggtgggggcagcagcagccgccccgcctcccactcccatgcccaccagcactcgcccatctcccctcggacccaggatgccctccagcagagacccagtgtgcttcacaacacaggcatgaagggtatcatcaccgctgtggagcccagcaagcccacggtcctgaggtccacctccacctcctcacccgttcgcccagctgccacattcccacctgccacccactgcccactgggcggcaccctcgatggggtctaccctaccctcatggagcccgtcttgctgcccaaggaggccccccgggtcgcccggccagagcggccccgagcagacaccggccatgccttcctcgccaagcccccagcccgctccgggctggagcccgcctcctcccccagcaagggctcggagccccggcccctagtgcctcctgtctctggccacgccaccatcgcccgcacccctgcgaagaacctcgcacctcaccacgccagcccggacccgccggcgccacctgcctcggcctcggacccgcaccgggaaaagactcaaagtaaacccttttccatccaggaactggaactccgttctctgggttaccacggcagcagctacagccccgaaggggtggagcccgtcagccctgtgagctcacccagtctgacccacgacaaggggctccccaagcacctggaagagctcgacaagagccacctggagggggagctgcggcccaagcagccaggccccgtgaagcttggcggggaggccgcccacctcccacacctgcggccgctgcctgagagccagccctcgtccagcccgctgctccagaccgccccaggggtcaaaggtcaccagcgggtggtcaccctggcccagcacatcagtgaggtcatcacacaggactacacccggcaccacccacagcagctcagcgcacccctgcccgcccccctctactccttccctggggccagctgccccgtcctggacctccgccgcccacccagtgacctctacctcccgcccccggaccatggtgccccggcccgtggctccccccacagcgaagggggcaagaggtctccagagccaaacaagacgtcggtcttgggtggtggtgaggacggtattgaacctgtgtccccaccggagggcatgacggagccagggcactcccggagtgctgtgtacccgctgctgtaccgggatggggaacagacggagcccagcaggatgggctccaagtctccaggcaacaccagccagccgccagccttcttcagcaagctgaccgagagcaactccgccatggtcaagtccaagaagcaagagatcaacaagaagctgaacacccacaaccggaatgagcctgaatacaatatcagccagcctgggacggagatcttcaatatgcccgccatcaccggaacaggccttatgacctatagaagccaggcggtgcaggaacatgccagcaccaacatggggctggaggccataattagaaaggcactcatgggtaaatatgaccagtgggaagagtccccgccgctcagcgccaatgcttttaaccctctgaatgccagtgccagcctgcccgctgctatgcccataaccgctgctgacggacggagtgaccacacactcacctcgccaggtggcggcgggaaggccaaggtctctggcagacccagcagccgaaaagccaagtccccggccccgggcctggcatctggggaccggccaccctctgtctcctcagtgcactcggagggagactgcaaccgccggacgccgctcaccaaccgcgtgtgggaggacaggccctcgtccgcaggttccacgccattcccctacaaccccctgatcatgcggctgcaggcgggtgtcatggcttccccacccccaccgggcctccccgcgggcagcgggcccctcgctggcccccaccacgcctgggacgaggagcccaagccactgctctgctcgcagtacgagacactctccgacagcgag

SEQ ID NO: 4 - DNA, 8544 bp, Mus musculus
  CDS (160)..(7545)
ctcgagcccgatccgccgtagcccggcgccagcgcccggtgccgccgccgggaggcacctgtgacgaggtcacctgccagcagatgaccgagaccagcccttagtcctaggtgtggtcaagagtgtcttggctccaaagcctacctggaccctaccaccatgtcaggatccacacagcctgtggcacagacatggcgggctgctgagccccgctacccaccccatggcatctcctacccggtgcagatagcccggtcccacacggacgtggggctgcttgagtaccaacaccacccccgtgactacacctcacacctgtcacccggttccatcatccagccacagaggaggcggccctcactgctgtcagagttccagcctgggagtgaacggtctcaggagctccacctgcgccctgagtcccgcacgttcctgcctgagctgggcaagcccgacatagaattcaccgagagcaagcgcccccgcctggagctactacccgataccctgctgcgcccatcacccctgctggccactgggcagccgagtgggtctgaagaccttaccaaggaccgtagcctggcaggcaagctggagcctgtgtcacctcccagtcccccgcacgctgaccctgagctagagctggcgccatctcgactgtccaaggaggagctgatccagaacagattggaccgcgtggaccgtgagatcaccatggtagagcagcagatctccaagctgaagaagaagcagcaacagttggaggaggaggccgccaagccgcccgaacccgagaagcctgtgtcgccaccacccatagaatcaaagcaccgaagcctggtccagatcatctacgatgagaaccggaagaaagccgaagccgcacaccggatcctagaaggcctggggccccaggtggagctgcctctgtacaaccagccgtctgacacacgccagtaccatgaaaacatcaaaataaaccaggcgatgcggaagaagctgatcttgtactttaagcggaggaaccacgcgcgcaagcagtgggaacagcgcttctgccagcgctatgaccagctcatggaggcgtgggagaagaaggtagagcgcatagagaacaatccgcgaaggagggccaaggagagcaaggtgagggagtactacgagaaacagttcccggagatccgcaagcagcgggagctgcaggagcgcatgcagagcagggtgggccagcgtggcagtgggctctccatgtcggctgcccgcagtgagcatgaggtttctgagatcattgatggcttgtctgagcaggagaacctggagaagcagatgcgccagctggccgtgatccgccatgttgtacgacgcgaccagcagaggatcaagttcatcaacatgaatggactcatggatgaccccatgaaggtctacaaggaccgtcaggttaccaacatgtggagcgagcaggagagggacaccttccgtgagaagtttatgcagcaccctaagaactttggcctgattgcctcattcctggagagaaagacggtcgctgagtgtgtcctctattactacctgaccaagaagaatgaaaattacaagagcttggtgaggcggagctatcggcgccgtggcaagagccagcagcagcagcagcagcaacaacagcagcagcagcagcagatggcacggagcagccaggaggagaaggaggagaaggagaaggagaaggaggccgacaaggaggaagagaagcaggatgcggagaacgagaaggaagaactcagcaaggagaagacagacgacacttctggcgaggacaacgatgagaaagaggccgtggcctccaaaggccgcaaaactgccaacagccaaggccgccgcaaaggccgtatcacgcgctccatggccaacgaggccaaccatgaggagacagccaccccacagcaaagttcagagctggcttccatggagatgaacgagagttctcgctggactgaggaagagatggagacagcaaagaaaggcctcctggaacatgggaggaactggtcagccattgcccgcatggtgggctccaagaccgtgtcccagtgtaagaacttctacttcaactacaagaagaggcagaacctggacgaaatccttcagcagcacaagctaaagatggagaaggagaggaacgctcggaggaagaagaagaagaccccagctgcggcgagcgaggagacagccttcccacctgccgctgaggacgaagagatggaagcatcaggcgcaagtgccaatgaggaagagctggcggaggaggcagaagcctcacaggcctctgggaatgaggttcccagagttggggagtgcagtggcccagctgctgtcaacaacagctctgatactgagagtgtcccatccccgcgttcagaagccacgaaggacactgggcctaaacccactggcactgaagcattgcccgctgccacccagccacctgttcctcctccagaagaaccggcagcagcccctgctgagccctccccagtccctgatgccagtggcccaccatccccagagccttccccatcacctgccgcacccccggctactgtggacaaggatgaacaagaagccccggctgctccagctccccagacagaagatgccaaggagcagaagtctgaggccgaggagatcgatgtgggaaagccagaggagcccgaggcctctgaggagcccccggagagtgtaaagagtgaccacaaggaggagaccgaggaagagcctgaagacaaagccaagggcacagaggccattgaaactgtgtctgaggcaccacttaaggtggaggaggctggtagcaaggcagctgtgaccaagggttccagctcaggtgccacccaggacagtgactccagtgccacctgcagtgccgatgaggtggacgaacccgaaggaggtgacaagggcaggctgctgtcaccaaggcccagcctcctcaccccggctggagatccccgggccagtacctcgccccagaagccgctggacctgaagcagctgaagcagcgagcagccgccatcccccctatcgtcaccaaggtccatgagcccccccgggaggacacagtacccccaaagccagttccccctgtgcctccacccacgcagcacctacagccagagggtgacgtgtctcagcagtcgggaggaagtccacgtggcaagtcccgcagcccagtgcctcctgccgagaaagaggcagagaaacccgcattctttccggctttcccaactgagggccaaagctaccgactgagcccccacgctggtcatcggctgccttcccatcctccacgggaggtgatcaagacttccacacgcgctgaccctctcttctcctacacaccccccggtcacccgctgcctctgggcctccacgatagtgcccggcccgtcctgccacgtccccccatctctaaccccccacccctcatctcctctgccaagcatcccggcgtacttgagaggcagctgggtgccatctcccaggggatgtcagtccagcttcgtgtgcctcactcagagcatgccaagcccatgggccctctcaccatggagctgccccttgccgtggaccctaagaagctggggacagcactggctccgccaccagtggaagcatcaccaagggcctcccagtacccgggctgcagacggccccagctacagaggctctatcacccacgcacgcccgcagacgtcctctacaagggtaccatcagcaggatcgtcggtgaggacagcccaagtcgccttgaccgggcacgagaggacaccctgcccaagggccatgtcatctatgagggcaagaaaggccacgtcctatcctatgaaggtggtatgtccgtgtcacagtgctctaaggaggatggaaggagcagctcgggcccaccccatgagactgccgcccctaaacgcacctatgacatgatggagggccgtgtaggcaggactgtcacctcagccagcatagagggactcatgggccgcgccatccctgagcagcacagcccccacctcaaggagcagcatcacatccgaggctccatcacgcaaggcatcccgaggtcctatgtggaggcgcaggaggactacttacggcgggaggccaagctcttgaagcgagaagggacaccacctcccccaccaccacctcgggacctgactgagacctacaagccccggcccctggaccctctgggtcccctgaagctgaagccgactcacgagggtgtggtagcaactgtgaaggaggcgggccgctctatccatgagatcccgagagaggagctgcgccgcacacctgagctacccctggcaccacggcctctgaaggagggttccatcacccagggcaccccactcaagtacgactctggggcaccctccactggcaccaagaaacacgacgtgcgctccatcatcggcagccccggccggcctttccctgccctgcacccgctggacataatggctgacgcccgggcactggagcgtgcctgctatgaagagagtctgaagagccggtcagggaccagcagtggtgcagggggctccatcacacgtggggctccagtcgtcgtgcctgaactgggcaagccacggcaaagcccactgacttacgaagaccacggggcacccttcaccagtcacctgccacgtggctcccctgtgaccacgagggagcccacgccacgccttcaggaaggcagcctcctatccagcaaggcgtcccaggaccggaagctgacatctacaccccgggagatcgccaagtccccacacagcactgtgcccgagcaccaccctcaccccatctccccctatgagcacttgctccggggcgtgactggtgtggacctgtaccgtggtcacatcccattggcctttgaccccacctccataccccgagggatccctctggaagcagcagccgcagcctactacctgccccggcacttggcccccagccccacctacccacacctgtacccaccttacctcatccgcggctaccctgacacggcggccctggagaaccgccagaccatcatcaatgactacatcacctcgcagcagatgcaccacaacgctgcctccgccatggcccagcgtgctgacatgctgaggggtctgtcaccgcgagagtcctcgctggccctcaattattccgctggcccaagaggcattatcgacctgtcccaagtgccacacctgcccgtgctggtgccaccaacgccaggcacccctgccaccgccatcgaccgccttgcctacctccccactgcgcccccacccttcagcagccgccacagtagctcaccgctgtccccaggaggccccactcacctagctaaaccaactgccacatcttcatcggagcgggaacgggaacgtgagcgggaacgagacaagtccatcctcacgtctaccactacagtggagcatgcacccatctggagacctggtacggagcagagcagcggggctgggggcagcagccgccccgcctcccacacccaccagcactcgcccatctccccccggacccaggacgccttgcagcagaggcccagtgtgctgcacaacacgagcatgaagggcgtggtcacctccgtggaacccggcacgcccacggtcctgaggtccacctccacctcttcgcctgtccgcccagctgccacattcccacctgccacccactgcccacttggtggcacccttgaaggggtctaccctaccctcatggagcccgtcctgttacccaaggagacctctcgggtcgcccggcccgagcggccccgtgtggacggtggccatgccttcctcaccaaacccccggcccgggagcccgcctcctcacccagcaagagctccgagccccgatccctagcaccccccagctccagccacacagccatcgcccgcaccccagcaaagagccttgcaccccaccatgccagtccggacccgccggggcccacctcggcctcagatctgcaccgagaaaagactcaaagtaaacccttttccatccaggaattggaactccgttctctgggttaccacagtggagctggctacagccccgatggggtggagcccatcagcccggtgagctcccccagcctgacccacgacaaggggctctccaaacctctggaagagctagagaagagccacttggaaggggagctgcggcacaagcagccaggccccatgaagctcagcgcggaggctgcccatctcccacatctgcggccactgcccgagagccagccctcatccagcccactcctccagactgccccaggcatcaaaggtcaccagagggtggtcaccctggctcagcacatcagcgaggtcattacgcaggactacacccggcaccacccgcagcagctcagtggcccccttcccgcccctctctactcctttcccggagccagctgccctgtgctggatcttcgccgcccacccagtgacctctacctcccaccccccgaccatggcaccccagcccggggatccccccacagtgaagggggcaaaaggtccccagaacccagcaaaacatcggtcctgggcagcagtgaggatgccattgagcctgtgtccccaccagagggcatgactgagccaggacatgctcggagcgctgtgtacccactgctgtatcgagacggggaacagggcgagcccaggatgggctctaagtctccaggcaacaccagccagccgccagccttcttcagtaagctgactgagagcaactccgccatggtgaagtcgaagaagcaggagatcaacaagaaactcaacacccacaaccggaacgagccagaatacaatattggccagcctgggacggaaatcttcaacatgcccgccatcactggagcaggccttatgacctgtagaagccaggcggtgcaagaacacgccagcaccaacatggggctagaggccattattagaaaggcactcatgggtaaatatgatcagtgggaagagcccccgccgctcggcgccaatgcttttaaccctctgaatgccagcgccagtctgcccgctgctgctatgcccataaccactgctgacggacggagtgaccacgcactcacctcgccaggtggaggtgggaaagccaaggtctctggcagacctagcagccgaaaagccaagtcgccagcaccaggcctagcgtccggagaccgacccccttctgtctcctcagtacactcagagggggactgcaatcgccgaacaccactcaccaaccgtgtgtgggaggaccggccctcatctgcagggtccacgccattcccctacaaccctttgattatgaggctacaggcaggtgtcatggcctccccgcccccacctggccttgcggcaggcagcgggcccctagctggtccccaccacgcctgggatgaggagcccaagccactgctgtgttcacagtatgagacactctcggacagcgagtgaccacggattgggggggagcggtgccaggtcccgcacaaggcagaagcagcccagcatggagcagacagctgctgactccggagactgaggaaggagcccctgagtctgcctgccgtccatccgtccgtccgtccactcatctgtccatccagagctggcatcctgcctgtctaaagccttaactaagactcccaccccgggctggccctgcgcagtgaccttacactcaggggatgtttacctggtgctcgagaggggagtggacaggaggggagggacagcgggccaggaggggggggacagcaatcgtgtgtcagtcgcactcgtgcatctggggtcagcggggacccaccacaggctgaccaggcacctccatgccaccgcctcgccccttaccgcatttggaaccaaagtctaactgaactctcgcgtggtcctgcccctctctctgcccccagcccgcttgcctctggacagacagacgttcccagcttatcctgccctaatgctgtcatcatcgcagtctccaaaggccacccagcccacaagactgggagcccatcagaccaggtgggtgacacaaggggcctggctggggcacggatgcttgcaggaactggaccgtttcccggcctgttgctgtggcaacgggagggaaggcacgtgtaaatggtgttggcttacagggtatatttttgataccttcaatgagttaattcagacgtctcacacaaggaaggactcgcccgtgtttctcccgctgtgctttggtctctacctactgtttcagaggcacgtgccagccaaggcggtggcccaccatacgcaggacttgggggtcaggggctccggcacacggcactgtgcccttccccacaccttacttcagcgaaatggacttgatgcgtattctgtggccgctctctgtgcacggcggcattctgtcatttacacatgttgttccaattaaaaagcaaatatactcaagtgaaaaaa

SEQ ID NO: 5 - protein, 2462 aa, Mus musculus
MSGSTQPVAQTWRAAEPRYPPHGISYPVQIARSHTDVGLLEYQHHPRDYTSHLSPGSIIQPQRRRPSLLSEFQPGSERSQELHLRPESRTFLPELGKPDIEFTESKRPRLELLPDTLLRPSPLLATGQPSGSEDLTKDRSLAGKLEPVSPPSPPHADPELELAPSRLSKEELIQNRLDRVDREITMVEQQISKLKKKQQQLEEEAAKPPEPEKPVSPPPIESKHRSLVQIIYDENRKKAEAAHRILEGLGPQVELPLYNQPSDTRQYHENIKINQAMRKKLILYFKRRNHARKQWEQRFCQRYDQLMEAWEKKVERIENNPRRRAKESKVREYYEKQFPEIRKQRELQERMQSRVGQRGSGLSMSAARSEHEVSEIIDGLSEQENLEKQMRQLAVIRHVVRRDQQRIKFINMNGLMDDPMKVYKDRQVTNMWSEQERDTFREKFMQHPKNFGLIASFLERKTVAECVLYYYLTKKNENYKSLVRRSYRRRGKSQQQQQQQQQQQQQQMARSSQEEKEEKEKEKEADKEEEKQDAENEKEELSKEKTDDTSGEDNDEKEAVASKGRKTANSQGRRKGRITRSMANEANHEETATPQQSSELASMEMNESSRWTEEEMETAKKGLLEHGRNWSAIARMVGSKTVSQCKNFYFNYKKRQNLDEILQQHKLKMEKERNARRKKKKTPAAASEETAFPPAAEDEEMEASGASANEEELAEEAEASQASGNEVPRVGECSGPAAVNNSSDTESVPSPRSEATKDTGPKPTGTEALPAATQPPVPPPEEPAAAPAEPSPVPDASGPPSPEPSPSPAAPPATVDKDEQEAPAAPAPQTEDAKEQKSEAEEIDVGKPEEPEASEEPPESVKSDHKEETEEEPEDKAKGTEAIETVSEAPLKVEEAGSKAAVTKGSSSGATQDSDSSATCSADEVDEPEGGDKGRLLSPRPSLLTPAGDPRASTSPQKPLDLKQLKQRAAAIPPIVTKVHEPPREDTVPPKPVPPVPPPTQHLQPEGDVSQQSGGSPRGKSRSPVPPAEKEAEKPAFFPAFPTEGQSYRLSPHAGHRLPSHPPREVIKTSTRADPLFSYTPPGHPLPLGLHDSARPVLPRPPISNPPPLISSAKHPGVLERQLGAISQGMSVQLRVPHSEHAKPMGPLTMELPLAVDPKKLGTALAPPPVEASPRASQYPGCRRPQLQRLYHPRTPADVLYKGTISRIVGEDSPSRLDRAREDTLPKGHVIYEGKKGHVLSYEGGMSVSQCSKEDGRSSSGPPHETAAPKRTYDMMEGRVGRTVTSASIEGLMGRAIPEQHSPHLKEQHHIRGSITQGIPRSYVEAQEDYLRREAKLLKREGTPPPPPPPRDLTETYKPRPLDPLGPLKLKPTHEGVVATVKEAGRSIHEIPREELRRTPELPLAPRPLKEGSITQGTPLKYDSGAPSTGTKKHDVRSIIGSPGRPFPALHPLDIMADARALERACYEESLKSRSGTSSGAGGSITRGAPVVVPELGKPRQSPLTYEDHGAPFTSHLPRGSPVTTREPTPRLQEGSLLSSKASQDRKLTSTPREIAKSPHSTVPEHHPHPISPYEHLLRGVTGVDLYRGHIPLAFDPTSIPRGIPLEAAAAAYYLPRHLAPSPTYPHLYPPYLIRGYPDTAALENRQTIINDYITSQQMHHNAASAMAQRADMLRGLSPRESSLALNYSAGPRGIIDLSQVPHLPVLVPPTPGTPATAIDRLAYLPTAPPPFSSRHSSSPLSPGGPTHLAKPTATSSSERERERERERDKSILTSTTTVEHAPIWRPGTEQSSGAGGSSRPASHTHQHSPISPRTQDALQQRPSVLHNTSMKGVVTSVEPGTPTVLRSTSTSSPVRPAATFPPATHCPLGGTLEGVYPTLMEPVLLPKETSRVARPERPRVDGGHAFLTKPPAREPASSPSKSSEPRSLAPPSSSHTAIARTPAKSLAPHHASPDPPGPTSASDLHREKTQSKPFSIQELELRSLGYHSGAGYSPDGVEPISPVSSPSLTHDKGLSKPLEELEKSHLEGELRHKQPGPMKLSAEAAHLPHLRPLPESQPSSSPLLQTAPGIKGHQRVVTLAQHISEVITQDYTRHHPQQLSGPLPAPLYSFPGASCPVLDLRRPPSDLYLPPPDHGTPARGSPHSEGGKRSPEPSKTSVLGSSEDAIEPVSPPEGMTEPGHARSAVYPLLYRDGEQGEPRMGSKSPGNTSQPPAFFSKLTESNSAMVKSKKQEINKKLNTHNRNEPEYNIGQPGTEIFNMPAITGAGLMTCRSQAVQEHASTNMGLEAIIRKALMGKYDQWEEPPPLGANAFNPLNASASLPAAAMPITTADGRSDHALTSPGGGGKAKVSGRPSSRKAKSPAPGLASGDRPPSVSSVHSEGDCNRRTPLTNRVWEDRPSSAGSTPFPYNPLIMRLQAGVMASPPPPGLAAGSGPLAGPHHAWDEEPKPLLCSQYETLSDSE

SEQ ID NO: 6 - DNA, 7386 bp, Mus musculus
  CDS (1)..(7386)
atgtcaggatccacacagcctgtggcacagacatggcgggctgctgagccccgctacccaccccatggcatctcctacccggtgcagatagcccggtcccacacggacgtggggctgcttgagtaccaacaccacccccgtgactacacctcacacctgtcacccggttccatcatccagccacagaggaggcggccctcactgctgtcagagttccagcctgggagtgaacggtctcaggagctccacctgcgccctgagtcccgcacgttcctgcctgagctgggcaagcccgacatagaattcaccgagagcaagcgcccccgcctggagctactacccgataccctgctgcgcccatcacccctgctggccactgggcagccgagtgggtctgaagaccttaccaaggaccgtagcctggcaggcaagctggagcctgtgtcacctcccagtcccccgcacgctgaccctgagctagagctggcgccatctcgactgtccaaggaggagctgatccagaacagattggaccgcgtggaccgtgagatcaccatggtagagcagcagatctccaagctgaagaagaagcagcaacagttggaggaggaggccgccaagccgcccgaacccgagaagcctgtgtcgccaccacccatagaatcaaagcaccgaagcctggtccagatcatctacgatgagaaccggaagaaagccgaagccgcacaccggatcctagaaggcctggggccccaggtggagctgcctctgtacaaccagccgtctgacacacgccagtaccatgaaaacatcaaaataaaccaggcgatgcggaagaagctgatcttgtactttaagcggaggaaccacgcgcgcaagcagtgggaacagcgcttctgccagcgctatgaccagctcatggaggcgtgggagaagaaggtagagcgcatagagaacaatccgcgaaggagggccaaggagagcaaggtgagggagtactacgagaaacagttcccggagatccgcaagcagcgggagctgcaggagcgcatgcagagcagggtgggccagcgtggcagtgggctctccatgtcggctgcccgcagtgagcatgaggtttctgagatcattgatggcttgtctgagcaggagaacctggagaagcagatgcgccagctggccgtgatccgccatgttgtacgacgcgaccagcagaggatcaagttcatcaacatgaatggactcatggatgaccccatgaaggtctacaaggaccgtcaggttaccaacatgtggagcgagcaggagagggacaccttccgtgagaagtttatgcagcaccctaagaactttggcctgattgcctcattcctggagagaaagacggtcgctgagtgtgtcctctattactacctgaccaagaagaatgaaaattacaagagcttggtgaggcggagctatcggcgccgtggcaagagccagcagcagcagcagcagcaacaacagcagcagcagcagcagatggcacggagcagccaggaggagaaggaggagaaggagaaggagaaggaggccgacaaggaggaagagaagcaggatgcggagaacgagaaggaagaactcagcaaggagaagacagacgacacttctggcgaggacaacgatgagaaagaggccgtggcctccaaaggccgcaaaactgccaacagccaaggccgccgcaaaggccgtatcacgcgctccatggccaacgaggccaaccatgaggagacagccaccccacagcaaagttcagagctggcttccatggagatgaacgagagttctcgctggactgaggaagagatggagacagcaaagaaaggcctcctggaacatgggaggaactggtcagccattgcccgcatggtgggctccaagaccgtgtcccagtgtaagaacttctacttcaactacaagaagaggcagaacctggacgaaatccttcagcagcacaagctaaagatggagaaggagaggaacgctcggaggaagaagaagaagaccccagctgcggcgagcgaggagacagccttcccacctgccgctgaggacgaagagatggaagcatcaggcgcaagtgccaatgaggaagagctggcggaggaggcagaagcctcacaggcctctgggaatgaggttcccagagttggggagtgcagtggcccagctgctgtcaacaacagctctgatactgagagtgtcccatccccgcgttcagaagccacgaaggacactgggcctaaacccactggcactgaagcattgcccgctgccacccagccacctgttcctcctccagaagaaccggcagcagcccctgctgagccctccccagtccctgatgccagtggcccaccatccccagagccttccccatcacctgccgcacccccggctactgtggacaaggatgaacaagaagccccggctgctccagctccccagacagaagatgccaaggagcagaagtctgaggccgaggagatcgatgtgggaaagccagaggagcccgaggcctctgaggagcccccggagagtgtaaagagtgaccacaaggaggagaccgaggaagagcctgaagacaaagccaagggcacagaggccattgaaactgtgtctgaggcaccacttaaggtggaggaggctggtagcaaggcagctgtgaccaagggttccagctcaggtgccacccaggacagtgactccagtgccacctgcagtgccgatgaggtggacgaacccgaaggaggtgacaagggcaggctgctgtcaccaaggcccagcctcctcaccccggctggagatccccgggccagtacctcgccccagaagccgctggacctgaagcagctgaagcagcgagcagccgccatcccccctatcgtcaccaaggtccatgagcccccccgggaggacacagtacccccaaagccagttccccctgtgcctccacccacgcagcacctacagccagagggtgacgtgtctcagcagtcgggaggaagtccacgtggcaagtcccgcagcccagtgcctcctgccgagaaagaggcagagaaacccgcattctttccggctttcccaactgagggccaaagctaccgactgagcccccacgctggtcatcggctgccttcccatcctccacgggaggtgatcaagacttccacacgcgctgaccctctcttctcctacacaccccccggtcacccgctgcctctgggcctccacgatagtgcccggcccgtcctgccacgtccccccatctctaaccccccacccctcatctcctctgccaagcatcccggcgtacttgagaggcagctgggtgccatctcccaggggatgtcagtccagcttcgtgtgcctcactcagagcatgccaagcccatgggccctctcaccatggagctgccccttgccgtggaccctaagaagctggggacagcactggctccgccaccagtggaagcatcaccaagggcctcccagtacccgggctgcagacggccccagctacagaggctctatcacccacgcacgcccgcagacgtcctctacaagggtaccatcagcaggatcgtcggtgaggacagcccaagtcgccttgaccgggcacgagaggacaccctgcccaagggccatgtcatctatgagggcaagaaaggccacgtcctatcctatgaaggtggtatgtccgtgtcacagtgctctaaggaggatggaaggagcagctcgggcccaccccatgagactgccgcccctaaacgcacctatgacatgatggagggccgtgtaggcaggactgtcacctcagccagcatagagggactcatgggccgcgccatccctgagcagcacagcccccacctcaaggagcagcatcacatccgaggctccatcacgcaaggcatcccgaggtcctatgtggaggcgcaggaggactacttacggcgggaggccaagctcttgaagcgagaagggacaccacctcccccaccaccacctcgggacctgactgagacctacaagccccggcccctggaccctctgggtcccctgaagctgaagccgactcacgagggtgtggtagcaactgtgaaggaggcgggccgctctatccatgagatcccgagagaggagctgcgccgcacacctgagctacccctggcaccacggcctctgaaggagggttccatcacccagggcaccccactcaagtacgactctggggcaccctccactggcaccaagaaacacgacgtgcgctccatcatcggcagccccggccggcctttccctgccctgcacccgctggacataatggctgacgcccgggcactggagcgtgcctgctatgaagagagtctgaagagccggtcagggaccagcagtggtgcagggggctccatcacacgtggggctccagtcgtcgtgcctgaactgggcaagccacggcaaagcccactgacttacgaagaccacggggcacccttcaccagtcacctgccacgtggctcccctgtgaccacgagggagcccacgccacgccttcaggaaggcagcctcctatccagcaaggcgtcccaggaccggaagctgacatctacaccccgggagatcgccaagtccccacacagcactgtgcccgagcaccaccctcaccccatctccccctatgagcacttgctccggggcgtgactggtgtggacctgtaccgtggtcacatcccattggcctttgaccccacctccataccccgagggatccctctggaagcagcagccgcagcctactacctgccccggcacttggcccccagccccacctacccacacctgtacccaccttacctcatccgcggctaccctgacacggcggccctggagaaccgccagaccatcatcaatgactacatcacctcgcagcagatgcaccacaacgctgcctccgccatggcccagcgtgctgacatgctgaggggtctgtcaccgcgagagtcctcgctggccctcaattattccgctggcccaagaggcattatcgacctgtcccaagtgccacacctgcccgtgctggtgccaccaacgccaggcacccctgccaccgccatcgaccgccttgcctacctccccactgcgcccccacccttcagcagccgccacagtagctcaccgctgtccccaggaggccccactcacctagctaaaccaactgccacatcttcatcggagcgggaacgggaacgtgagcgggaacgagacaagtccatcctcacgtctaccactacagtggagcatgcacccatctggagacctggtacggagcagagcagcggggctgggggcagcagccgccccgcctcccacacccaccagcactcgcccatctccccccggacccaggacgccttgcagcagaggcccagtgtgctgcacaacacgagcatgaagggcgtggtcacctccgtggaacccggcacgcccacggtcctgaggtccacctccacctcttcgcctgtccgcccagctgccacattcccacctgccacccactgcccacttggtggcacccttgaaggggtctaccctaccctcatggagcccgtcctgttacccaaggagacctctcgggtcgcccggcccgagcggccccgtgtggacggtggccatgccttcctcaccaaacccccggcccgggagcccgcctcctcacccagcaagagctccgagccccgatccctagcaccccccagctccagccacacagccatcgcccgcaccccagcaaagagccttgcaccccaccatgccagtccggacccgccggggcccacctcggcctcagatctgcaccgagaaaagactcaaagtaaacccttttccatccaggaattggaactccgttctctgggttaccacagtggagctggctacagccccgatggggtggagcccatcagcccggtgagctcccccagcctgacccacgacaaggggctctccaaacctctggaagagctagagaagagccacttggaaggggagctgcggcacaagcagccaggccccatgaagctcagcgcggaggctgcccatctcccacatctgcggccactgcccgagagccagccctcatccagcccactcctccagactgccccaggcatcaaaggtcaccagagggtggtcaccctggctcagcacatcagcgaggtcattacgcaggactacacccggcaccacccgcagcagctcagtggcccccttcccgcccctctctactcctttcccggagccagctgccctgtgctggatcttcgccgcccacccagtgacctctacctcccaccccccgaccatggcaccccagcccggggatccccccacagtgaagggggcaaaaggtccccagaacccagcaaaacatcggtcctgggcagcagtgaggatgccattgagcctgtgtccccaccagagggcatgactgagccaggacatgctcggagcgctgtgtacccactgctgtatcgagacggggaacagggcgagcccaggatgggctctaagtctccaggcaacaccagccagccgccagccttcttcagtaagctgactgagagcaactccgccatggtgaagtcgaagaagcaggagatcaacaagaaactcaacacccacaaccggaacgagccagaatacaatattggccagcctgggacggaaatcttcaacatgcccgccatcactggagcaggccttatgacctgtagaagccaggcggtgcaagaacacgccagcaccaacatggggctagaggccattattagaaaggcactcatgggtaaatatgatcagtgggaagagcccccgccgctcggcgccaatgcttttaaccctctgaatgccagcgccagtctgcccgctgctgctatgcccataaccactgctgacggacggagtgaccacgcactcacctcgccaggtggaggtgggaaagccaaggtctctggcagacctagcagccgaaaagccaagtcgccagcaccaggcctagcgtccggagaccgacccccttctgtctcctcagtacactcagagggggactgcaatcgccgaacaccactcaccaaccgtgtgtgggaggaccggccctcatctgcagggtccacgccattcccctacaaccctttgattatgaggctacaggcaggtgtcatggcctccccgcccccacctggccttgcggcaggcagcgggcccctagctggtccccaccacgcctgggatgaggagcccaagccactgctgtgttcacagtatgagacactctcggacagcgag

(19) United States
(12) Patent Application Publication
(10) Pub. No.: US 2003/0027137 A1
(43) Pub. Date: Feb. 6, 2003

(54) Novel nuclear receptor corepressor molecules and uses therefor

(76) Inventor: J. Don Chen, Westboro, MA (US)

Correspondence Address: LAHIVE & COCKFIELD, 28 STATE STREET, BOSTON, MA, 02109, US

(21) Appl. No.: 09/819,104

(22) Filed: Mar. 27, 2001

Related U.S. Application Data
(60) Provisional application No. 60/193,138, filed on Mar. 29, 2000.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/358; 536/23.5

(57) ABSTRACT

The invention provides isolated nucleic acids molecules, designated SMRTe nucleic acid molecules, which encode proteins involved in nuclear receptor mediated activities. The invention also provides antisense nucleic acid molecules, recombinant expression vectors containing SMRTe nucleic acid molecules, host cells into which the expression vectors have been introduced, methods for identifying compounds that modulate SMRTe activity, and nonhuman transgenic animals in which a SMRTe gene has been introduced or disrupted. The invention still further provides isolated SMRTe proteins, fusion proteins, antigenic peptides, and anti-SMRTe antibodies. Diagnostic methods utilizing compositions of the invention are also provided.

### RELATED INFORMATION

This application claims priority to U.S. provisional Application No. 60/193,138, entitled “NOVEL NUCLEAR RECEPTOR COREPRESSOR MOLECULES AND USES THEREFOR,” filed on Mar. 29, 2000, incorporated herein in its entirety by this reference. The contents of the sequence listing, figures, patents, patent applications, and references cited throughout this specification are hereby incorporated by reference in their entireties.

### BACKGROUND OF THE INVENTION

Transcriptional repression of gene expression plays an important role in the proper regulation of cell growth, differentiation, and development (Johnson et al. (1995) Cell 81, 655-658; Hanna-Rose et al. (1996) Trends Genet. 12, 229-234; and DePinho et al. (1998) Nature 391, 535-536). In one mechanism of transcriptional inhibition of gene expression, a repressor competes with an activator for DNA binding. Alternatively, transcriptional repressors also can inhibit basal transcription of gene expression through direct interaction with general transcription factors, or indirectly by promoting chromatin condensation, thereby preventing the loading of general transcription factors to the promoter necessary for expression of a particular gene.

Transcriptional repression by nuclear receptors such as thyroid hormone receptor (TR) and retinoic acid receptor (RAR) play important roles in the regulation of cell growth, differentiation, and homeostasis. In the absence of hormone, TR and RAR actively repress target gene expression by interacting with the corepressors termed silencing mediator for retinoid and thyroid hormone receptors (SMRT) and nuclear receptor corepressor (N-CoR), which are components of corepressor complexes that also contain mSin3A/B and histone deacetylases (Horlein et al. (1995) Nature 377, 397-404; Nagy et al. (1997) Cell 89, 373-380; Alland et al. (1997) Nature 387, 49-55; Heinzel et al (1997) Nature 387, 43-48). Corepressors help to prevent gene expression until the binding of hormone to the corresponding receptor causes dissociation of the corepressor leading to transcriptional activation of gene expression (Baniahmad et al. (1992) Cell 11, 1015-1023; Renaud et al. (1995) Nature 378, 681-689; Rastinejad et al. (1995) Nature 375, 203-211; Bourguet, W., Ruff, M., Chambon, P., Gronemeyer, H. & Moras, D. (1995) Nature (London) 375, 377-382; Chen et al. (1998) Crit. Rev. Eukaryot. Gene Exp. 8, 169-190).

In addition to TR and RAR, other transcriptional regulators are now known to be involved in a wide array of biological processes (including, e.g., leukemogenesis) and signaling pathways that are modulated by corepressors including, e.g., the orphan nuclear receptors (e.g., COUP-TF1, Rev-Erb, RVR), and DAX-1), the progesterone and estrogen receptors, promyelocyte zinc finger protein PLZF, the acute myeloid leukemia fusion partner ETO, as well as several non-nuclear receptor proteins such as the homeodomain proteins Rpx2, Pit-1, and the mammalian homologue of Drosophila Suppressor of Hairless CBF 1/RBP-Jkappa which is involved in Notch signaling (Shibata et al (1997) Mol. Endocrinol. 11, 714-724; Zamir et al. (1996) Mol. Cell. Biol. 16, 5458-5465; Crawford et al.(1998) Mol. Cell. Biol. 18, 2949-2956; Muscatelli et al. (1994) Nature 372, 672-676; Wagner et al. (1998) Mol. Cell. Biol. 18,1369-1378; Zhang et al. (1998) Mol. Endocrinol. 12, 513-524; He et al. (1998) Nat. Genet. 18,126-135; Hong et al. (1997) PNAS 94, 9028-9033; Wong et al. (1998) J. Biol. Chem. 273, 27695-27702; Lin et al. (1998) Nature 391, 811-814; Westendorf et al. (1998) Mol. Cell. Biol. 18, 322-333; Lutterbach et al. (1998) Mol. Cell. Biol. 18, 7176-7184; Grignanai et al. (1998) Nature 391, 815-818; Gelmetti et al. (1998) Mol. Cell. Biol. 18, 7185-7191; Xu et al. (1998) Nature 395, 301-306; and Kao et al. (1998) Genes Dev. 12, 2269-2277).

Given the importance of corepressors in the modulation of a wide variety of signaling pathways and biological processes, there exists a need for the identification of novel corepressor molecules and modulators thereof, in particular, for use in modulating gene transcription regulated by nuclear receptor family members.

### SUMMARY OF THE INVENTION

The present invention is based, at least in part, on the discovery of novel SMRT nuclear receptor corepressor family members containing an extended region (e), referred to herein as “SMRTe proteins” (“SMRTe”) nucleic acid and protein molecules. The SMRTe molecules of the present invention are useful as targets for discovering and developing modulating agents to regulate a variety of cellular processes. Accordingly, in one aspect, the invention provides isolated nucleic acid molecules encoding SMRTe proteins or biologically active portions thereof, as well as nucleic acid fragments suitable as primers or hybridization probes for the detection of SMRTe-encoding nucleic acids.

In one embodiment, a SMRTe nucleic acid molecule of the invention is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to the nucleotide sequence (e.g., to the entire length of the nucleotide sequence) shown in SEQ ID NO:1, SEQ ID NO:3, or a complement thereof. In another embodiment, a SMRTe nucleic acid molecule of the invention is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to the nucleotide sequence (e.g., to the entire length of the nucleotide sequence) shown in SEQ ID NO:4, SEQ ID NO:6, or a complement thereof.

In a preferred embodiment, the isolated nucleic acid molecule includes the nucleotide sequence shown in SEQ ID NO:1 or a complement thereof. In another embodiment, the nucleic acid molecule includes SEQ ID NO:3 and nucleotides 1-156 of SEQ ID NO:1. In another embodiment, the nucleic acid molecule includes SEQ ID NO:3 and nucleotides 7681-8686 of SEQ ID NO:1. In another preferred embodiment, the nucleic acid molecule has the nucleotide sequence shown in SEQ ID NO: 3. In another preferred embodiment, the nucleic acid molecule includes a fragment of at least 50 nucleotides of the nucleotide sequence of SEQ ID NO:1, SEQ ID NO:3, or a complement thereof.

In a preferred embodiment, the isolated nucleic acid molecule includes the nucleotide sequence shown in SEQ ID NO: 6, or a complement thereof. In another embodiment, the nucleic acid molecule includes SEQ ID NO:6 and nucleotides 1-159 of SEQ ID NO:4. In another embodiment, the nucleic acid molecule includes SEQ ID NO:6 and nucleotides 7549-8544 of SEQ ID NO:4. In another preferred embodiment, the nucleic acid molecule has the nucleotide sequence shown in SEQ ID NO: 6. In another preferred embodiment, the nucleic acid molecule includes a fragment of at least 50 nucleotides of the nucleotide sequence of SEQ ID NO:4, SEQ ID NO:6, or a complement thereof.

In another preferred embodiment, the isolated nucleic acid molecule includes at least 25 consecutive nucleotides, more preferably at least 50 consecutive nucleotides, more preferably at least 100 consecutive nucleotides, more preferably at least 200 consecutive nucleotides, more preferably at least 400 consecutive nucleotides, more preferably at least 600 consecutive nucleotides, more preferably at least 800 consecutive nucleotides, more preferably at least 1000 consecutive nucleotides, more preferably at least 1200 consecutive nucleotides, more preferably at least 1400 consecutive nucleotides, more preferably at least 1600, more preferably at least 2000, more preferably at least 3000, more preferably at least 4000, more preferably at least 5000, more preferably at least 6000, more preferably at least 7000, more preferably at least 8500 consecutive nucleotides of the nucleotide sequence shown in SEQ ID NO: 1 or 3, or a complement thereof.

In another preferred embodiment, the isolated nucleic acid molecule includes at least 25 consecutive nucleotides, more preferably at least 50 consecutive nucleotides, more preferably at least 100 consecutive nucleotides, more preferably at least 200 consecutive nucleotides, more preferably at least 400 consecutive nucleotides, more preferably at least 600 consecutive nucleotides, more preferably at least 800 consecutive nucleotides, more preferably at least 1000 consecutive nucleotides, more preferably at least 1200 consecutive nucleotides, more preferably at least 1400 consecutive nucleotides, more preferably at least 1600, more preferably at least 2000, more preferably at least 3000, more preferably at least 4000, more preferably at least 5000, more preferably at least 6000, more preferably at least 7000, more preferably at least 8500 consecutive nucleotides of the nucleotide sequence shown in SEQ ID NO:4 or SEQ ID NO:6, or a complement thereof.

In another embodiment, a SMRTe nucleic acid molecule includes a nucleotide sequence encoding a protein having an amino acid sequence sufficiently homologous to the amino acid sequence of SEQ ID NO:2, or SEQ ID NO:5. In a preferred embodiment, a SMRTe nucleic acid molecule includes a nucleotide sequence encoding a protein having an amino acid sequence at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to the amino acid sequence of SEQ ID NO:2 or SEQ ID NO:5.

In another preferred embodiment, an isolated nucleic acid molecule encodes the amino acid sequence of human or murine SMRTe. In yet another preferred embodiment, the nucleic acid molecule includes a nucleotide sequence encoding a protein having the amino acid sequence of SEQ ID NO:2 or SEQ ID NO:5. In yet another preferred embodiment, the nucleic acid molecule is at least 300 nucleotides in length and encodes a protein having a SMRTe activity (as described herein).

Another embodiment of the invention features nucleic acid molecules, preferably SMRTe nucleic acid molecules, which specifically detect SMRTe nucleic acid molecules relative to nucleic acid molecules encoding non-SMRTe proteins. For example, in one embodiment, such a nucleic acid molecule is at least 50, 60, 70, 80, 90, 100, 150, 200, 300, 400, 500, 500-1000, 1000-1500, 1500-2000, 2000-2500, 2500-3000, 3000-4000, 4000-5000, 6000-7000, 7000-8000, or more nucleotides in length and/or hybridizes under stringent conditions to a nucleic acid molecule comprising the nucleotide sequence shown in SEQ ID NO:1, 4, or a complement thereof. It should be understood that the nucleic acid molecule can be of a length within a range having one of the numbers listed above as a lower limit and another number as the upper limit for the number of nucleotides in length, e.g., molecules that are 60-80, 300-1000, or 150-400 nucleotides in length. In preferred embodiments, the nucleic acid molecules (e.g., oligonucleotides or probes) are at least 15 (e.g., contiguous) nucleotides in length and hybridize under stringent conditions to nucleotides 157-7680 of SEQ ID NO:1. In other preferred embodiments, the nucleic acid molecules comprise nucleotides 160-7548 of SEQ ID NO:4.

In other preferred embodiments, the nucleic acid molecule encodes a naturally occurring allelic variant of a polypeptide comprising the amino acid sequence of SEQ ID NO:2, wherein the nucleic acid molecule hybridizes to a nucleic acid molecule comprising SEQ ID NO:1 or 3 under stringent conditions. In other preferred embodiments, the nucleic acid molecule encodes a naturally occurring allelic variant of a polypeptide comprising the amino acid sequence of SEQ ID NO:5, wherein the nucleic acid molecule hybridizes to a complement of a nucleic acid molecule comprising SEQ ID NO:4 or 6 under stringent conditions. Another embodiment of the invention provides an isolated nucleic acid molecule which is antisense to an SMRTe nucleic acid molecule, e.g., to the coding strand of a SMRTe nucleic acid molecule.

Another aspect of the invention provides a vector comprising a SMRTe nucleic acid molecule. In certain embodiments, the vector is a recombinant expression vector. n another embodiment, the invention provides a host cell containing a vector of the invention. The invention also provides a method for producing a protein, preferably a SMRTe protein, by culturing in a suitable medium, a host cell, e.g., a mammalian host cell such as a non-human mammalian cell, of the invention containing a recombinant expression vector, such that the protein is produced.

Another aspect of the invention features isolated or recombinant SMRTe proteins and polypeptides. In one embodiment, the isolated protein, preferably a SMRTe protein, includes an SNC domain, preferably, a biologically active portion of an SNC domain. In another embodiment, the isolated protein, preferably a SMRTe protein, contains one or more domains selected from the group consisting of a SANT domain (A and/or B), a polyglutamine track, a charged acidic-basic region, a highly conserved region between SMRTe and N-CoR, a SIT motif, a KGH motif, a serine/glycine-rich region, a SMRTe repression domain (SRD), and a nuclear receptor interacting domain (RID). In a preferred embodiment, the foregoing domains are biologically active.

In another preferred embodiment, the isolated protein includes at least 50 consecutive amino acids, more preferably at least 100 consecutive amino acids, more preferably at least 150 consecutive amino acids, more preferably at least 200 consecutive amino acids, more preferably at least 250 consecutive amino acids, more preferably at least 350 consecutive amino acids, more preferably at least 450 consecutive amino acids, more preferably at least 500 consecutive amino acids, more preferably at least 600 consecutive amino acids, more preferably at least 700 consecutive amino acids, more preferably at least 800 consecutive amino acids, more preferably at least 900 consecutive amino acids, more preferably at least 1000 consecutive amino acids, more preferably at least 1500 consecutive amino acids, more preferably at least 2000 consecutive amino acids, more preferably at least 2500 consecutive amino acids or more of the amino acid sequence shown SEQ ID NO:2 or SEQ ID NO:5.

In another embodiment, the invention features fragments of the proteins having the amino acid sequence of SEQ ID NO:2 or SEQ ID NO:5 wherein the fragment comprises at least 15 amino acids (e.g., contiguous amino acids) of the amino acid sequence of SEQ ID NO:2 or SEQ ID NO:5. In another embodiment, the protein, preferably a SMRTe protein, has the amino acid sequence of SEQ ID NO:2 or SEQ ID NO:5.

In another embodiment, the invention features an isolated protein, preferably a SMRTe protein, which is encoded by a nucleic acid molecule having a nucleotide sequence at least about 50%, 55%, 60%, 65%, 70%, 75%, 80%, 85%, 90%, 95% or more homologous to a nucleotide sequence of SEQ ID NO: 1, SEQ ID NO:3, or a complement thereof. In yet another embodiment, the invention features an isolated protein, preferably a SMRTe protein, which is encoded by a nucleic acid molecule having a nucleotide sequence at least about 50%, 55%, 60%, 65%, 70%, 75%, 80%, 85%, 90%, 95% or more homologous to a nucleotide sequence of SEQ ID NO:4, SEQ ID NO:6, or a complement thereof.

The proteins of the present invention or biologically active portions thereof, can be operatively linked to a non-SMRTe polypeptide (e.g., heterologous amino acid sequences) to form fusion proteins. The invention further features antibodies, such as monoclonal or polyclonal antibodies, that specifically bind proteins of the invention, preferably SMRTe proteins. In addition, the SMRTe proteins or biologically active portions thereof can be incorporated into pharmaceutical compositions, which optionally include pharmaceutically acceptable carriers.

In another aspect, the present invention provides a method for detecting the presence of a SMRTe nucleic acid molecule, protein or polypeptide in a biological sample by contacting the biological sample with an agent capable of detecting a SMRTe nucleic acid molecule, protein or polypeptide such that the presence of a SMRTe nucleic acid molecule, protein or polypeptide is detected in the biological sample.

In another aspect, the present invention provides a method for detecting the presence of SMRTe activity in a biological sample by contacting the biological sample with an agent capable of detecting an indicator of SMRTe activity such that the presence of SMRTe activity is detected in the biological sample.

In another aspect, the invention provides a method for modulating SMRTe activity comprising contacting a cell capable of expressing SMRTe with an agent that modulates SMRTe activity such that SMRTe activity in the cell is modulated. In one embodiment, the agent inhibits SMRTe activity. In another embodiment, the agent stimulates SMRTe activity. In one embodiment, the agent is an antibody that specifically binds to a SMRTe protein. In another embodiment, the agent modulates expression of SMRTe by modulating transcription of a SMRTe gene or translation of a SMRTe mRNA. In yet another embodiment, the agent is a nucleic acid molecule having a nucleotide sequence that is antisense to the coding strand of a SMRTe mRNA or a SMRTe gene.

In one embodiment, the methods of the present invention are used to treat a subject having a disorder characterized by aberrant SMRTe protein or nucleic acid expression or activity by administering an agent which is a SMRTe modulator to the subject. In one embodiment, the SMRTe modulator is a SMRTe protein. In another embodiment the SMRTe modulator is a SMRTe nucleic acid molecule. In yet another embodiment, the SMRTe modulator is a peptide, peptidomimetic, or other small molecule. In a preferred embodiment, the disorder characterized by aberrant SMRTe protein or nucleic acid expression is a cancer.

The present invention also provides a diagnostic assay for identifying the presence or absence of a genetic alteration characterized by at least one of (i) aberrant modification or mutation of a gene encoding a SMRTe protein; (ii) mis-regulation of the gene; and (iii) aberrant post-translational modification of a SMRTe protein, wherein a wild-type form of the gene encodes an protein with a SMRTe activity.

In another aspect the invention provides a method for identifying a compound that binds to or modulates the activity of a SMRTe protein, by providing an indicator composition comprising a SMRTe protein having SMRTe activity, contacting the indicator composition with a test compound, and determining the effect of the test compound on SMRTe activity in the indicator composition to identify a compound that modulates the activity of a SMRTe protein.

Other features and advantages of the invention will be apparent from the following detailed description and claims.

### BRIEF DESCRIPTION OF THE DRAWINGS

FIG. 1 shows a comparison of the amino acid sequences of human (h) SMRTe (upper strand; see also SEQ ID NO: 2) and murine (m) SMRTe (bottom strand; see also SEQ ID NO: 5) (sequence identity indicated by hyphens; dots are gaps introduced during the alignment). The COOH-terminal tail of the mSMRTeC, the starting amino acids of the previously identified SMRT, and TRAC1, are also indicated.

FIG. 2 shows an autoradiograph and immunoblots indicating the presence of endogenous SMRT and related SMRTe proteins in a mammalian nuclear cell (HeLa) extract. One major polypeptide similar to the size of N-CoR (270 kDa) was detected in the HeLa nuclear extract, in addition to two minor bands of 180 and 80 kDa (arrows).

FIG. 3 shows a domain comparison between SMRTe and N-CoR. The black bars indicate areas of high homology. Special domains are indicated in gray with labels (AB, acidic-basic domain; S1-4, the SIT repeated motifs; KGH, the KGH repeated motifs; SG, the serine/glycine-rich region; and SNC). The SMRTe repression domains (SRD), the N-CoR repression domains (NRD), and the nuclear receptor interacting domains (RID) are also shown. Domains involved in interactions with other proteins are also indicated. The numbering of residues is based on mouse N-CoR and human SMRTe sequence.

FIG. 4 shows a comparison of the SNC domains of human (h) and mouse (m) SMRTe (S) and N-CoR (N). Identical residues are shown in black and the conserved residues are shown in gray. The amphipathic helix and the hydrophobic heptad repeats are indicated by a black line and stars, respectively. The amino acid residues are shown on the left. The lower panel shows a comparison of SANT-A and SANT-B domains. Identical amino acids are shown in black background and the conserved residues are in gray. The Myb DNA binding domain signature sequences and the three helices (h) are also indicated in between the SANT-A and SANT-B motifs.

FIG. 5 shows a schematic of different SMRTe domains (panel A) tested for functional activity in a transcriptional repression assay (panel B). The SMRTe domains are as described in FIG. 3 and the text and numbers indicate amino acid residues. The seven different SMRTe N-terminal fragments (A to G) were fused to the Gal4 DNA-binding domain and their effects on reporter gene expression were assayed (B). The fold repression of each construct was determined by average relative luciferase activity using a Gal4 DNA-binding domain as a standard in a triplicate experiment.

FIG. 6 shows photographs (panels A and B) and an immunoblot (panel C) depicting cell cycle-dependent expression patterns of SMRTe. Panel A shows immunofluorescence staining of endogenous SMRTe in HeLa cells (lower) and overall nuclear staining using DAPI (upper). Panel B shows immunostaining of SMRTe in an unsynchronized population of A549 cells. Panel C shows an immunoblot for SMRTe in A549 cells at different time points after release from mitosis.

FIG. 7 shows photomicrographs indicating the distribution of SMRTe transcripts in a mouse embryo at different developmental stages. In particular, SMRTe transcripts were detected by in situ hybridization in thin sections of (Panel A) embryonic day (E)9.0 days post conception, (Panel B) E11.5, and (Panel C) E13.5 using a DIG-labeled antisense riboprobe. Panels c1 and c2 show enlargement of areas in the cartilage and lung at E13.5 indicated by rectangles in Panel C. Panel D shows the control background signal using a DIG-labeled sense probe. Abbreviations are: b, brain; ba, bronchial arch; br, bronchus; c, cartilage; cp, cerebellar plate; h, heart; im, limb; lu, lung; lv, liver; nt, neural tube; pc, perichondrium; sc, sclerotome; vb, vertebra body.

### DETAILED DESCRIPTION OF THE INVENTION

The present invention is based, at least in part, on the discovery of novel, human and murine transcriptional corepressors that interact with nuclear hormone receptors from both human and mouse. These novel corepressors contain over 1,000 addition amino acid residues at the N-terminal of protein sequence related to the human silencing mediator for retinoid and thyroid hormone receptors or SMRT protein. Accordingly, the SMRT family members of the invention having a novel extended region (e) and are referred to herein as SMRTe nucleic acids and proteins.

The identification of SMRTe reveals an unexpected similarity between SMRT and N-CoR, a related nuclear receptor corepressor. SMRT and N-CoR function as transcriptional corepressors for nuclear hormone receptors. And transcriptional repression of gene expression plays an important role in the proper regulation of cell growth, differentiation, and development (Johnson et al. (1995) Cell 81, 655-658; Hanna-Rose et al. (1996) Trends Genet. 12, 229-234; and DePinho et al. (1998) Nature 391, 535-536).

Accordingly, the SMRTe molecules of the invention are suitable targets for developing novel diagnostic targets and therapeutic agents to control gene regulation in a number of different cell types. Moreover, the SMRTe molecules of the invention are suitable targets for developing diagnostic targets and therapeutic agents for detecting and/or treating cells or tissues having misregulated gene expression that occur, e.g., in a cancer (see also U.S. Ser. No. 08/522,726; Ordentlich et al. (1999) PNAS 6,2639-2644).

In particular, the novel human SMRTe molecules described herein, can have one or more of the following activities:

(i) regulation of TR and/or RAR; (ii) and thus are useful as (1) targets for the development of new strategies for altering retinoid or thyroid hormone-mediated gene regulation, and (2) as targets for the development of new strategies for altering gene regulation that can contribute, e.g., to a cancer pathology such as acute promyelocytic leukemia (APL) and breast cancer;

(ii) regulation of other transcriptional regulators involved in a wide array of biological processes (including, e.g., leukemogenesis); and

(iii) regulation of signaling pathways that are modulated by corepressors, including, e.g., the orphan nuclear receptors (e.g., COUP-TF1, Rev-Erb, RVR), and DAX-1), the progesterone and estrogen receptors, promyelocyte zinc finger protein PLZF, the acute myeloid leukemia fusion partner ETO, Mad/Max proteins, and STATs.

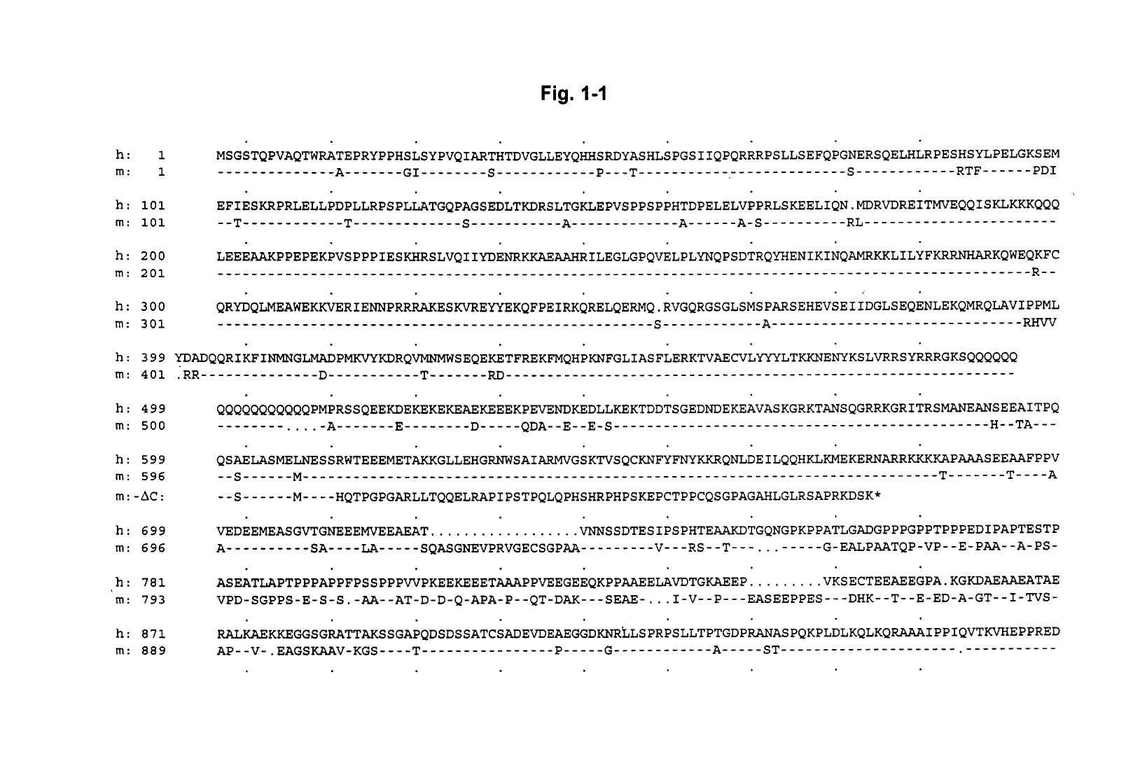

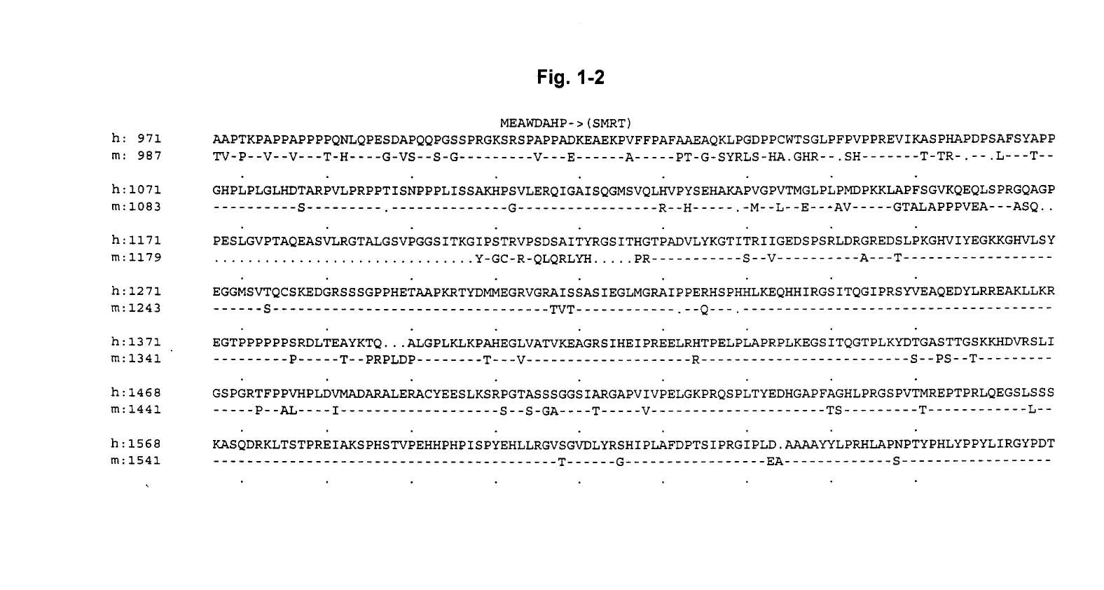

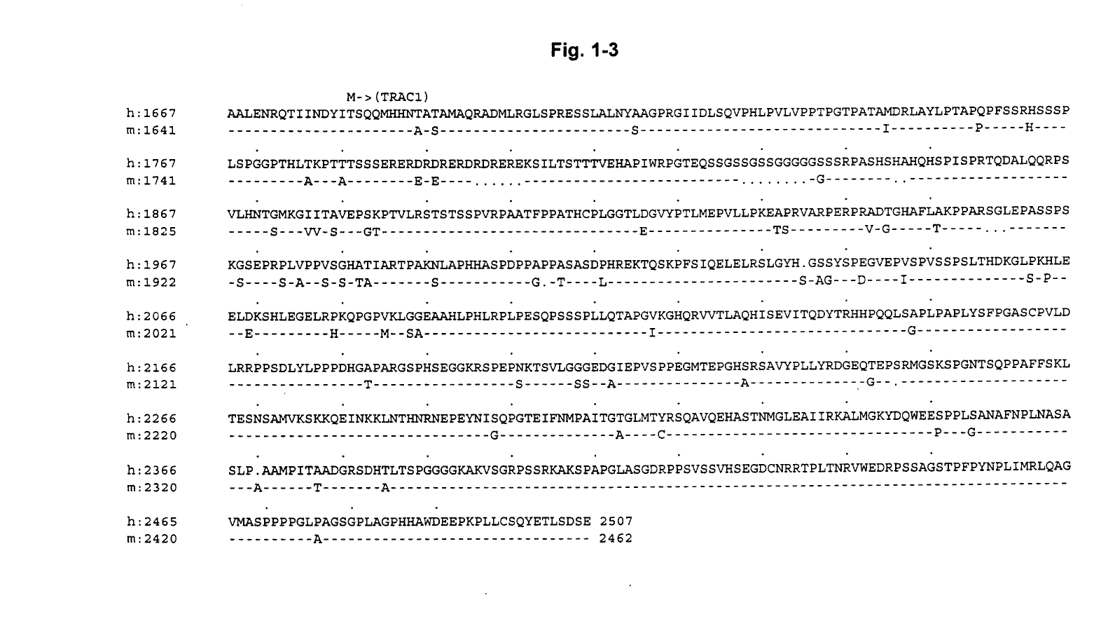

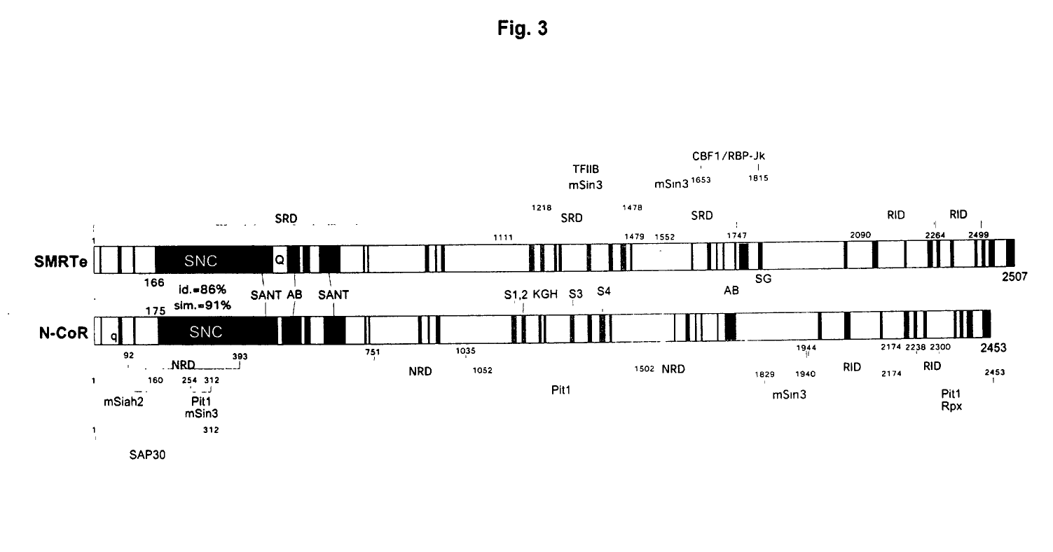

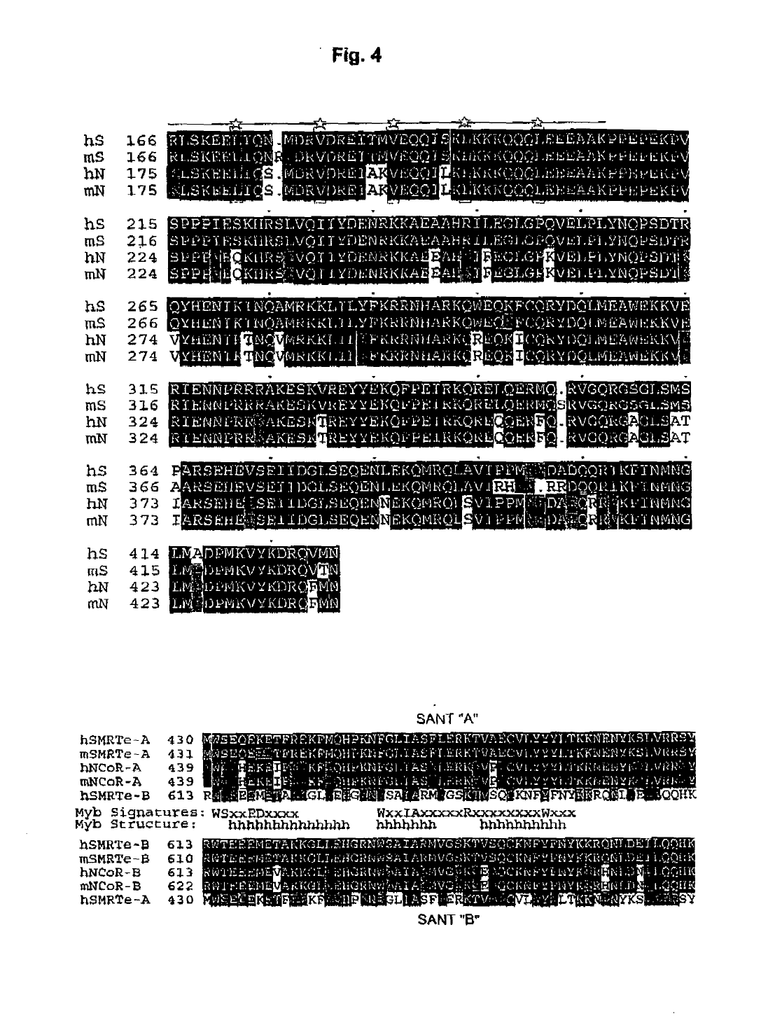

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin. An N-terminal domain between amino acid residues 166 and 429 is conserved between SMRTe and N-CoR (86% identity and 91% similarity) (see, e.g., FIG. 1). Accordingly, this domain was termed the SMRTe and N-CoR conserved (SNC) domain. The SNC domain was determined to have at the N terminus an amphipathic-helix containing five hydrophobic heptad repeats (FIG. 4). Thus, the family of SMRTe proteins comprise at least one functional domain such as SNC domain and preferably at least one other protein domain such as, e.g., a SANT domain. In addition, members of a family may also have common functional characteristics such as corepressor activity, i.e., SMRTe activity.

The term “SANT domain” refers to conserved repeats known as the SANT (SWI3, ADA2, N-CoR, and TFIIIB B″) domains (Aasland et al. (1996) Trends Biochem. Sci. 21, 87-88) and these domains typically follow the SNC domain. The two SANT motifs of the SMRTe proteins are only marginally related to one another within the same protein (30% identity), whereas the individual motif is highly conserved between SMRTe and N-CoR in both the human and mouse (>75% identity) (FIG. 4). Therefore, the N-terminal SANT domain is referred to as SANT-A and the C-terminal domain as SANT-B (FIG. 4). The SANT-A and SANT-B domain are separated by an intervening sequence of approximately 120 amino acids, which contains a polyglutamine track and a charged acidic-basic region followed by a short segment that also is highly conserved between SMRTe and N-CoR (FIG. 1). Accordingly, another SMRTe domain may comprise a polyglutamine track and, optionally, a charged acidic-basic region followed by a short segment that is highly conserved between SMRTe and N-CoR.

Other characteristic SMRTe domains include SIT repeated motifs, KGH repeated motifs, a serine/glycine-rich region, SMRTe repression domains (SRD), and nuclear receptor interacting domains (RID) and these are indicated in FIG. 3 (see also Li et al. (1997) Mol. Endocrinol. 11, 2025-2037).

Isolated proteins of the present invention, preferably SMRTe proteins, have an amino acid sequence sufficiently homologous to the amino acid sequence of SEQ ID NO: 2 or 5 and are encoded by a nucleotide sequence sufficiently homologous to SEQ ID NO: 1 or 4. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30% homology, preferably 40%-50%, preferably 60%-70%, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 30% homology, preferably 40%-50%, preferably 60%-70%, more preferably 70%-80%, and even more preferably 90-95% homology and share a common functional activity are defined herein as sufficiently homologous.

As used interchangeably herein, “SMRTe activity”, “biological activity of SMRTe” or “functional activity of SMRTe”, refers to an activity exerted by a SMRTe protein, polypeptide, or nucleic acid molecule on an SMRTe responsive cell or on an SMRTe protein substrate, as determined in vitro, or in vitro, according to standard techniques. Preferably, an SMRTe activity has the ability to act as a repressor or corepressor of gene transcription and these terms may be used interchangeably.

In one embodiment, SMRTe activity is a direct activity, such as an association with a transcriptional regulator and/or repression of gene transcription. In another embodiment, the SMRT activity is the ability of the polypeptide to modulate the function of other proteins involved in gene regulation, promoter activation, chromatin condensation, and/or acetylation or deacetylation of proteins involved in these activities such as, e.g., transcriptional regulators, TATA-binding proteins (TBP) associated factors (TAFs), thyroid hormone associated proteins (TRAPs), and/or histones.

Accordingly, another embodiment of the invention features isolated SMRTe proteins and polypeptides having a SMRTe activity. Preferred proteins are SMRTe proteins having a SNC domain, preferably one or more SMRTe related domains as described above, and, preferably, a SMRTe activity. The nucleotide sequence of the isolated human and murine SMRTe nucleic acids, cDNAs, and the predicted amino acid sequence of the SMRTe proteins encoded thereby are shown in SEQ ID NOs: 1-6 and FIG. 1.

The human SMRTe gene, which is approximately 8686 nucleotides in length, encodes a protein having a molecular weight of approximately 270 kDa and which is approximately 2507 amino acid residues in length.

The murine SMRTe gene, which is approximately 8544 nucleotides in length, encodes a protein having a molecular weight of approximately 270 kDa and which is approximately 2462 amino acid residues in length.

Various aspects of the invention are described in further detail in the following subsections:

I. Isolated Nucleic Acid Molecules

One aspect of the invention pertains to isolated nucleic acid molecules that encode SMRTe proteins or biologically active portions thereof, as well as nucleic acid fragments sufficient for use as hybridization probes to identify SMRTe-encoding nucleic acid molecules (e.g., SMRTe mRNA) and fragments for use as PCR primers for the amplification or mutation of SMRTe nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SMRTe nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO: 1 or 3, or a portion thereof, can be isolated using standard molecular biology techniques and the sequence information provided herein. In addition, a nucleic acid molecule of the present invention, e.g., a nucleic acid molecule having the nucleotide sequence of SEQ ID NO: 4 or 6, or a portion thereof, can be isolated using standard molecular biology techniques and the sequence information provided herein. Using all or portion of the nucleic acid sequence of SEQ ID NO: 1, 3, 4, or 6 as a hybridization probe, SMRTe nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NO: 1, 3, 4, or 6 can be isolated by the polymerase chain reaction (PCR) using synthetic oligonucleotide primers designed based upon the sequence of SEQ ID NO: 1, 3, 4, or 6.

A nucleic acid of the invention can be amplified using cDNA, mRNA, or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SMRTe nucleotide sequences can be prepared by standard synthetic techniques, e.g., using an automated DNA synthesizer. In a preferred embodiment, an isolated nucleic acid molecule of the invention comprises the nucleotide sequence shown in SEQ ID NO: 1. The sequence of SEQ ID NO: 1 corresponds to the human SMRTe cDNA. This cDNA comprises sequences encoding the human SMRTe protein (i.e., “the coding region”, from nucleotides 157-7677, as well as 5′ untranslated sequences (nucleotides 1-156) and 3′ untranslated sequences (nucleotides 7678-8686). Alternatively, the nucleic acid molecule can comprise only the coding region of SEQ ID NO: 1 (e.g., nucleotides 157-7677, corresponding to SEQ ID NO: 3).

In addition, the invention also encompasses the sequence of SEQ ID NO: 4 which corresponds to the murine SMRTe cDNA. This cDNA comprises sequences encoding the human SMRTe protein (i.e., “the coding region”, from nucleotides 160-7545, as well as 5′ untranslated sequences (nucleotides 1-159) and 3′ untranslated sequences (nucleotides 7546-8544). Alternatively, the nucleic acid molecule can comprise only the coding region of SEQ ID NO: 4 (e.g., nucleotides 157-7677, corresponding to SEQ ID NO: 6).

In another preferred embodiment, an isolated nucleic acid molecule of the invention comprises a nucleic acid molecule which is a complement of the nucleotide sequence shown in SEQ ID NO: 1, 3, 4, or 6, or a portion of any of these nucleotide sequences. A nucleic acid molecule which is complementary to the nucleotide sequence shown in SEQ ID NO: 1, 3, 4, or 6, is one which is sufficiently complementary to the nucleotide sequence shown in SEQ ID NO: 1, 3, 4, or 6, such that it can hybridize to the nucleotide sequence shown in SEQ ID NO: 1, 3, 4, or 6, thereby forming a stable duplex.

In still another preferred embodiment, an isolated nucleic acid molecule of the present invention comprises a nucleotide sequence which is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to the entire length of the nucleotide sequence shown in SEQ ID NO: 1, 3, 4, or 6, or a portion of any of these nucleotide sequences.

Moreover, the nucleic acid molecule of the invention can comprise only a portion of the nucleic acid sequence of SEQ ID NO: 1, 3, 4, or 6, for example, a fragment which can be used as a probe or primer or a fragment encoding a portion of an SMRTe protein, e.g., a biologically active portion of an SMRTe protein. The nucleotide sequence determined from the cloning of the SMRTe gene allows for the generation of probes and primers designed for use in identifying and/or cloning other SMRTe family members, as well as SMRTe homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO: 1, 3, 4, or 6, or of an anti-sense sequence of SEQ ID NO: 1, 3, 4, or 6, or of a naturally occurring allelic variant or mutant of SEQ ID NO: 1, 3, 4, or 6. In an exemplary embodiment, a nucleic acid molecule of the present invention comprises a nucleotide sequence which is greater than 50, 60, 70, 80, 90, 100, 150, 200, 300, 400, 500-1000, 1000-1500, 1500-2000, 2000-2500, 2500-3000, 3000-4000, 5000-6000, 6000-7000, 7000-8000, or more nucleotides in length and hybridizes under stringent hybridization conditions to a complement of a nucleic acid molecule of SEQ ID NO: 1, 3, 4, or 6.

Probes based on the SMRTe nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a SMRTe protein, such as by measuring a level of an SMRTe-encoding nucleic acid in a sample of cells from a subject e.g., detecting SMRTe mRNA levels or determining whether a genomic SMRTe gene has been mutated or deleted.

A nucleic acid fragment encoding a “biologically active portion of an SMRTe protein” can be prepared by isolating a portion of the nucleotide sequence of SEQ ID NO: 1, 3, 4, or 6, which encodes a polypeptide having an SMRTe biological activity (the biological activities of the SMRTe proteins are described herein), expressing the encoded portion of the SMRTe protein (e.g., by recombinant expression in vitro) and assessing the activity of the encoded portion of the SMRTe protein.

The invention further encompasses nucleic acid molecules that differ from the nucleotide sequence shown in SEQ ID NO: 1, 3, 4, or 6, due to degeneracy of the genetic code and thus encode the same SMRTe proteins as those encoded by the nucleotide sequence shown in SEQ ID NO: 1, 3, 4, or 6. In another embodiment, an isolated nucleic acid molecule of the invention has a nucleotide sequence encoding a protein having an amino acid sequence shown in SEQ ID NO: 2 or 5.

In addition to the SMRTe nucleotide sequences shown in SEQ ID NO: 1, 3, 4, or 6, it will be appreciated by those skilled in the art that DNA sequence polymorphisms that lead to changes in the amino acid sequences of the SMRTe proteins may exist within a population (e.g., the human population). Such genetic polymorphism in the SMRTe genes may exist among individuals within a population due to natural allelic variation. As used herein, the terms “gene” and “recombinant gene” refer to nucleic acid molecules which include an open reading frame encoding an SMRTe protein, preferably a mammalian SMRTe protein, and can further include non-coding regulatory sequences, and introns.

Allelic variants of human SMRTe include both functional and non-functional SMRTe proteins. Functional allelic variants are naturally occurring amino acid sequence variants of the human SMRTe that maintain the ability to bind a SMRTe ligand, e.g., a nuclear hormone receptor. Functional allelic variants will typically contain only conservative substitution of one or more amino acids of SEQ ID NO: 2 or 5 or substitution, deletion, or insertion of non-critical residues in non-critical regions of the protein.

Non-functional allelic variants are naturally occurring amino acid sequence variants of the human SMRTe protein that do not have the ability to either bind a SMRTe ligand, e.g., a nuclear hormone receptor. Non-functional allelic variants will typically contain a non-conservative substitution, a deletion, or insertion or premature truncation of the amino acid sequence of SEQ ID NO: 2 or a substitution, insertion or deletion in critical residues or critical regions.

The present invention further provides non-human orthologues of the human SMRTe protein. Orthologues of the human SMRTe protein are proteins that are isolated from non-human organisms and possess the same SMRTe activity of the human SMRTe protein such as, e.g., murine SMRTe. Orthologues of the human SMRTe protein can readily be identified as comprising an amino acid sequence that is substantially homologous to SEQ ID NO: 2 (compare to SEQ ID NO: 5; see also FIG. 1).

Moreover, nucleic acid molecules encoding other SMRTe family members and, thus, which have a nucleotide sequence which differs from the SMRTe sequences of SEQ ID NO: 1, 3, 4, or 6, are intended to be within the scope of the invention. For example, another SMRTe cDNA can be identified based on the nucleotide sequence of the human SMRTe or murine SMRTe. Moreover, nucleic acid molecules encoding SMRTe proteins from different species, e.g, mammals, and which, thus, have a nucleotide sequence which differs from the SMRTe sequences of SEQ ID NO: 1, 3, 4, or 6 are intended to be within the scope of the invention. For example, a rat or primate SMRTe cDNA can be identified based on the nucleotide sequence of the murine or human SMRTe.

Nucleic acid molecules corresponding to natural allelic variants and homologues of the SMRTe cDNAs of the invention can be isolated based on their homology to the SMRTe nucleic acids disclosed herein using the cDNAs disclosed herein, or a portion hereof, as a hybridization probe according to standard hybridization techniques under stringent hybridization conditions. Nucleic acid molecules corresponding to natural allelic variants and homologues of the SMRTe cDNAs of the invention can further be isolated by mapping to the same chromosome or locus as the SMRTe gene.

Accordingly, in another embodiment, an isolated nucleic acid molecule of the invention is at least 15, 20, 25, 30 or more nucleotides in length and hybridizes under stringent conditions to a complement of the nucleic acid molecule comprising the nucleotide sequence of SEQ ID NO: 1, 3, 4, or 6. In other embodiment, the nucleic acid is at least 30, 50, 100, 150, 200, 300, 400, 500, 600, 700, 800, 900, 1000, 1250, 1500, 1750, 2000, 2250, 2500, 3000, 4000, 5000, 6000, 7000, 8000, or more nucleotides in length. As used herein, the term “hybridizes under stringent conditions” is intended to describe conditions for hybridization and washing under which nucleotide sequences at least 50% homologous to each other typically remain hybridized to each other. Preferably, the conditions are such that sequences at least about 60%, even more preferably at least about 70%, 75%, 80%, 85%, 86%, 87%, 88%, 89%, 90%, 91%, 92%, 93%, 94%, 95%, 96%, 97%, 98%, 99%, or more homologous to each other typically remain hybridized to each other. Such stringent conditions are known to those skilled in the art and can be found in Current Protocols in Molecular Biology, John Wiley & Sons, N.Y. (1989), 6.3.1-6.3.6.

A preferred, non-limiting example of stringent hybridization conditions are hybridization in 6× sodium chloride/sodium citrate (SSC) at about 45° C., followed by one or more washes in 0.2 × SSC, 0.1% SDS at 50° C., preferably at 55° C., more preferably at 60° C., and even more preferably at 65° C. Preferably, an isolated nucleic acid molecule of the invention that hybridizes under stringent conditions to a complement of the sequence of SEQ ID NO: 1, 3, 4, or 6, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the SMRTe sequences that may exist in the population, the skilled artisan will further appreciate that changes can be introduced by mutation into the nucleotide sequences of SEQ ID NO: 1 or 3, thereby leading to changes in the amino acid sequence of the encoded SMRTe proteins, without altering the functional ability of the SMRTe proteins. For example, nucleotide substitutions leading to amino acid substitutions at “non-essential” amino acid residues can be made in the sequence of SEQ ID NO: 1 or 3. A “non-essential” amino acid residue is a residue that can be altered from the wild-type sequence of SMRTe (e.g., the sequence of SEQ ID NO: 2) without altering the biological activity, whereas an “essential” amino acid residue is required for biological activity.

Accordingly, another aspect of the invention pertains to nucleic acid molecules encoding SMRTe proteins that contain changes in amino acid residues that are not essential for activity. Such SMRTe proteins differ in amino acid sequence from SEQ ID NO: 2 (or SEQ ID NO:5), yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to the amino acid sequence of SEQ ID NO: 2 or 5.

An isolated nucleic acid molecule encoding an SMRTe protein homologous to the protein of SEQ ID NO: 2 or 5 can be created by introducing one or more nucleotide substitutions, additions, or deletions into the nucleotide sequence of, respectively, SEQ ID NO: 1 or 3, or, SEQ ID NO: 4 or 6 such that one or more amino acid substitutions, additions, or deletions are introduced into the encoded protein. Mutations can be introduced into SEQ ID NO: 1, 3, 4, or 6 by standard techniques, such as site-directed mutagenesis and PCR-mediated mutagenesis. Preferably, conservative amino acid substitutions are made at one or more predicted non-essential amino acid residues.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an SMRTe protein is preferably replaced with another amino acid residue from the same side chain family.

Alternatively, in another embodiment, mutations can be introduced randomly along all or part of a SMRTe coding sequence, such as by saturation mutagenesis, and the resultant mutants can be screened for SMRTe biological activity to identify mutants that retain activity. Following mutagenesis of SEQ ID NO: 1, 3, 4, or 6 the encoded protein can be expressed recombinantly and the activity of the protein can be determined.

In a preferred embodiment, a mutant SMRTe protein can be assayed for the ability to interact with a non-SMRTe molecule, e.g., a SMRTe ligand, e.g., a polypeptide or a small molecule.

In addition to the nucleic acid molecules encoding SMRTe proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire SMRTe coding strand, or to only a portion thereof.

In one embodiment, an antisense nucleic acid molecule is antisense to a “coding region” of the coding strand of a nucleotide sequence encoding SMRTe. The term “coding region” refers to the region of the nucleotide sequence comprising codons which are translated into amino acid residues (e.g., the coding region of human SMRTe corresponds to SEQ ID NO: 3).

In another embodiment, the antisense nucleic acid molecule is antisense to a “noncoding region” of the coding strand of a nucleotide sequence encoding SMRTe. The term “noncoding region” refers to 5′ and 3′ sequences which flank the coding region that are not translated into amino acids (i.e., also referred to as 5′ and 3′ untranslated regions).

Given the coding strand sequences encoding SMRTe disclosed herein (e.g., SEQ ID NO: 3), antisense nucleic acids of the invention can be designed according to the rules of Watson and Crick base pairing. The antisense nucleic acid molecule can be complementary to the entire coding region of SMRTe mRNA, but more preferably is an oligonucleotide which is antisense to only a portion of the coding or noncoding region of SMRTe mRNA. For example, the antisense oligonucleotide can be complementary to the region surrounding the translation start site of SMRTe mRNA. An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an SMRTe protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site.

Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330). In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SMRTe mRNA transcripts to thereby inhibit translation of SMRTe mRNA. A ribozyme having specificity for an SMRTe-encoding nucleic acid can be designed based upon the nucleotide sequence of an SMRTe cDNA disclosed herein (i.e., SEQ ID NO: 1). For example, a derivative of a Tetrahymena L-19 IVS RNA can be constructed in which the nucleotide sequence of the active site is complementary to the nucleotide sequence to be cleaved in an SMRTe-encoding mRNA (see, e.g., Cech et al. U.S. Pat. No. 4,987,071; and Cech et al U.S. Pat. No. 5,116,742). Alternatively, SMRTe mRNA can be used to select a catalytic RNA having a specific ribonuclease activity from a pool of RNA molecules. See, e.g., Bartel, D. and Szostak, J. W. (1993) Science 261:1411-1418.

Alternatively, SMRTe gene expression can be inhibited by targeting nucleotide sequences complementary to the regulatory region of the SMRTe (e.g., the SMRTe promoter and/or enhancers) to form triple helical structures that prevent transcription of the SMRTe gene in target cells. See generally, Helene, C. (1991) Anticancer Drug Des. 6(6):569-84; Helene, C. et al. (1992) Ann. N.Y Acad. Sci. 660:27-36; and Maher, L. J. (1992) Bioassays 14(12):807-15.

In yet another embodiment, the SMRTe nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of SMRTe nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of SMRTe nucleic acid molecules can also be used in the analysis of single base pair mutations in a gene, (e.g, by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of SMRTe nucleic acid molecules can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of SMRTe nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vitro), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g, PCT Publication No. WO89/10134). In addition, oligonucleotides can be modified with hybridization-triggered cleavage agents (See, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

II. Isolated SMRTe Proteins and Anti-SMRTe Antibodies

One aspect of the invention pertains to isolated SMRTe proteins, and biologically active portions thereof, as well as polypeptide fragments suitable for use as immunogens to raise anti-SMRTe antibodies. In one embodiment, native SMRTe proteins can be isolated from cells or tissue sources by an appropriate purification scheme using standard protein purification techniques. In another embodiment, SMRTe proteins are produced by recombinant DNA techniques. Alternative to recombinant expression, a SMRTe protein or polypeptide can be synthesized chemically using standard peptide synthesis techniques.

An “isolated” or “purified” protein or biologically active portion thereof is substantially free of cellular material or other contaminating proteins from the cell or tissue source from which the SMRTe protein is derived, or substantially free from chemical precursors or other chemicals when chemically synthesized. The language “substantially free of cellular material” includes preparations of SMRTe protein in which the protein is separated from cellular components of the cells from which it is isolated or recombinantly produced. In one embodiment, the language “substantially free of cellular material” includes preparations of SMRTe protein having less than about 30% (by dry weight) of non-SMRTe protein (also referred to herein as a “contaminating protein”), more preferably less than about 20% of non-SMRTe protein, still more preferably less than about 10% of non-SMRTe protein, and most preferably less than about 5% of non-SMRTe protein. When the SMRTe protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SMRTe protein in which the protein is separated from chemical precursors or other chemicals which are involved in the synthesis of the protein. In one embodiment, the language “substantially free of chemical precursors or other chemicals” includes preparations of SMRTe protein having less than about 30% (by dry weight) of chemical precursors or non-SMRTe chemicals, more preferably less than about 20% chemical precursors or non-SMRTe chemicals, still more preferably less than about 10% chemical precursors or non-SMRTe chemicals, and most preferably less than about 5% chemical precursors or non-SMRTe chemicals.

As used herein, a “biologically active portion” of an SMRTe protein includes a fragment of an SMRTe protein which participates in an interaction between an SMRTe molecule and a non-SMRTe molecule. Biologically active portions of an SMRTe protein include peptides comprising amino acid sequences sufficiently homologous to or derived from the amino acid sequence of the SMRTe protein, e.g., the amino acid sequence shown in SEQ ID NO: 2 (or SEQ ID NO: 5), which include less amino acids than the full length SMRTe proteins, and exhibit at least one activity of an SMRTe protein. Typically, biologically active portions comprise a domain or motif with at least one activity of the SMRTe protein. A biologically active portion of an SMRTe protein can be a polypeptide which is, for example, 10, 25, 50, 100, 200, 300, 400, 500, 600, 700,800,900, 1000, 1100, 1200, 1300, 1400, 1500, 1600, 1700, 1800, 1900,2000, 2100, 2200, 2300, 2400, 2500, or more amino acids in length. Biologically active portions of an SMRTe protein can be used as targets for developing agents which modulate a SMRTe mediated activity.

In one embodiment, a biologically active portion of an SMRTe protein comprises an SNC domain. Another preferred biologically active portion of an SMRTe protein may contain a SANT domain, a polyglutamine track, a charged acidic-basic region, a highly conserved region between SMRTe and N-CoR, a SIT motif, KGH motif, a serine/glycine-rich region, a SMRTe repression domain (SRD), and/or a nuclear receptor interacting domain (RID) and these are indicated in FIG. 3. Identification of these domains may be facilitated using any of a number of art recognized molecular modeling techniques as described herein (see also Example 1). Moreover, other biologically active portions, in which other regions of the protein are deleted, can be prepared by recombinant techniques and evaluated for one or more of the functional activities of a native SMRTe protein.

In a preferred embodiment, the SMRTe protein has an amino acid sequence shown in SEQ ID NO: 2 or 5. In other embodiments, the SMRTe protein is substantially homologous to SEQ ID NO: 2 or 5, and retains the functional activity of the protein of SEQ ID NO: 2 or 5, yet differs in amino acid sequence due to natural allelic variation or mutagenesis, as described in detail in subsection I above. Accordingly, in another embodiment, the SMRTe protein is a protein which comprises an amino acid sequence at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more homologous to SEQ ID NO: 2 or 5.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SMRTe nucleic acid molecules of the invention. BLAST protein searches can be performed with the XBLAST program, score=50, wordlength=3 to obtain amino acid sequences homologous to SMRTe protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) Comput. Appl. Biosci. 4:11-17. Such an algorithm is incorporated into the ALIGN program available, for example, at the GENESTREAM network server, IGH Montpellier, FRANCE (http://vega.igh.cnrs.fr) or at the ISREC server (http://www.ch.embnet.org). When utilizing the ALIGN program for comparing amino acid sequences, a PAM120 weight residue table, a gap length penalty of 12, and a gap penalty of 4 can be used.

The invention also provides SMRTe chimeric or fusion proteins. As used herein, a SMRTe “chimeric protein” or “fusion protein” comprises a SMRTe polypeptide operatively linked to a non-SMRTe polypeptide. A “SMRTe polypeptide” refers to a polypeptide having an amino acid sequence corresponding to SMRTe, whereas a “non-SMRTe polypeptide” refers to a polypeptide having an amino acid sequence corresponding to a protein which is not substantially homologous to the SMRTe protein, e.g., a protein which is different from the SMRTe protein and which is derived from the same or a different organism. Within a SMRTe fusion protein the SMRTe polypeptide can correspond to all or a portion of a SMRTe protein. In a preferred embodiment, a SMRTe fusion protein comprises at least one biologically active portion of a SMRTe protein. In another preferred embodiment, a SMRTe fusion protein comprises at least two biologically active portions of a SMRTe protein. Within the fusion protein, the term “operatively linked” is intended to indicate that the SMRTe polypeptide and the non-SMRTe polypeptide are fused in-frame to each other. The non-SMRTe polypeptide (e.g., a DNA binding domain) can be fused to the N-terminus or C-terminus of the SMRTe polypeptide (see Example 3).

For example, in one embodiment, the fusion protein is a GST-SMRTe fusion protein in which the SMRTe sequences are fused to the C-terminus of the GST sequences. Such fusion proteins can facilitate the purification of recombinant SMRTe.

In another embodiment, the fusion protein is a SMRTe protein containing a heterologous signal sequence at its N-terminus. In certain host cells (e.g., mammalian host cells), expression and/or secretion of SMRTe can be increased through use of a heterologous signal sequence.

Moreover, the SMRTe-fusion proteins of the invention can be used as immunogens to produce anti-SMRTe antibodies in a subject, to purify SMRTe ligands (e.g., protein partners) and in screening assays to identify molecules which inhibit the interaction of SMRTe with a SMRTe substrate.

Preferably, a SMRTe chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A SMRTe-encoding nucleic acid can be cloned into such an expression vector such that the fusion moiety is linked in-frame to the SMRTe protein.

The present invention also pertains to variants of the SMRTe proteins which function as either SMRTe agonists (mimetics) or as SMRTe antagonists. Variants of the SMRTe proteins can be generated by mutagenesis, e.g., discrete point mutation or truncation of a SMRTe protein. An agonist of the SMRTe proteins can retain substantially the same, or a subset, of the biological activities of the naturally occurring form of a SMRTe protein. An antagonist of a SMRTe protein can inhibit one or more of the activities of the naturally occurring form of the SMRTe protein by, for example, competitively modulating the corepressor activity of a SMRTe protein. Thus, specific biological effects can be elicited by treatment with a variant of limited function In one embodiment, treatment of a subject with a variant having a subset of the biological activities of the naturally occurring form of the protein has fewer side effects in a subject relative to treatment with the naturally occurring form of the SMRTe protein.

In one embodiment, variants of a SMRTe protein which function as either SMRTe agonists (mimetics) or as SMRTe antagonists can be identified by screening combinatorial libraries of mutants, e.g., truncation mutants, of a SMRTe protein for SMRTe protein agonist or antagonist activity. In one embodiment, a variegated library of SMRTe variants is generated by combinatorial mutagenesis at the nucleic acid level and is encoded by a variegated gene library. A variegated library of SMRTe variants can be produced by, for example, enzymatically ligating a mixture of synthetic oligonucleotides into gene sequences such that a degenerate set of potential SMRTe sequences is expressible as individual polypeptides, or alternatively, as a set of larger fusion proteins (e.g., for phage display) containing the set of SMRTe sequences therein. There are a variety of methods which can be used to produce libraries of potential SMRTe variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SMRTe sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a SMRTe protein coding sequence can be used to generate a variegated population of SMRTe fragments for screening and subsequent selection of variants of a SMRTe protein. In one embodiment, a library of coding sequence fragments can be generated by treating a double stranded PCR fragment of a SMRTe coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the SMRTe protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SMRTe proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a technique which enhances the frequency of functional mutants in the libraries, can be used in combination with the screening assays to identify SMRTe variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated SMRTe library. For example, a library of expression vectors can be transfected into a cell line which ordinarily synthesizes SMRTe. The transfected cells are then cultured such that SMRTe and a particular mutant SMRTe are expressed and the effect of expression of the mutant on SMRTe activity in the cells can be detected, e.g., by any of a number of enzymatic assays or by detecting an alteration in gene regulation using, e.g., a reporter gene. Plasmid DNA can then be recovered from the cells which score for inhibition, or alternatively, potentiation of SMRTe activity, and the individual clones further characterized.

An isolated SMRTe protein, or a portion or fragment thereof, can be used as an immunogen to generate antibodies that bind SMRTe using standard techniques for polyclonal and monoclonal antibody preparation. A full-length SMRTe protein can be used or, alternatively, the invention provides antigenic peptide fragments of SMRTe for use as immunogens. The antigenic peptide of SMRTe comprises at least 8 amino acid residues of the amino acid sequence shown in SEQ ID NO:2 or 5 and encompasses an epitope of SMRTe such that an antibody raised against the peptide forms a specific immune complex with SMRTe. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of SMRTe that are located on the surface of the protein, e.g., hydrophilic regions, as well as regions with high antigenicity.

A SMRTe immunogen typically is used to prepare antibodies by immunizing a suitable subject, (e.g., rabbit, goat, mouse, or other mammal) with the immunogen. An appropriate immunogenic preparation can contain, for example, recombinantly expressed SMRTe protein or a chemically synthesized SMRTe polypeptide. The preparation can further include an adjuvant, such as Freund's complete or incomplete adjuvant, or similar immunostimulatory agent. Immunization of a suitable subject with an immunogenic SMRTe preparation induces a polyclonal anti-SMRTe antibody response. Accordingly, another aspect of the invention pertains to anti-SMRTe antibodies. The term “antibody” as used herein refers to immunoglobulin molecules and immunologically active portions of immunoglobulin molecules, i.e., molecules that contain an antigen binding site which specifically binds (immunoreacts with) an antigen, such as SMRTe. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind SMRTe. The term “monoclonal antibody” or “monoclonal antibody composition”, as used herein, refers to a population of antibody molecules that contain only one species of an antigen binding site capable of immunoreacting with a particular epitope of SMRTe. A monoclonal antibody composition thus typically displays a single binding affinity for a particular SMRTe protein with which it immunoreacts.

Polyclonal anti-SMRTe antibodies can be prepared as described above by immunizing a suitable subject with a SMRTe immunogen. The anti-SMRTe antibody titer in the immunized subject can be monitored over time by standard techniques, such as with an enzyme linked immunosorbent assay (ELISA) using immobilized SMRTe. If desired, the antibody molecules directed against SMRTe can be isolated from the mammal (e.g., from the blood) and further purified by well known techniques, such as protein A chromatography to obtain the IgG fraction. At an appropriate time after immunization, e.g., when the anti-SMRTe antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) Proc. Natl. Acad. Sci. USA 76:2927-31; and Yeh et al. (1982) Int. J Cancer 29:269-75), the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a SMRTe immunogen as described above, and the culture supernatants of the resulting hybridoma cells are screened to identify a hybridoma producing a monoclonal antibody that binds SMRTe.

Any of the many well known protocols used for fusing lymphocytes and immortalized cell lines can be applied for the purpose of generating an anti-SMRTe monoclonal antibody (see, e.g., G. Galfre et al. (1977) Nature 266:55052; Gefter et al. Somatic Cell Genet., cited supra; Lerner, Yale J Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin, and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind SMRTe, e.g., using a standard ELISA assay.

Alternative to preparing monoclonal antibody-secreting hybridomas, a monoclonal anti-SMRTe antibody can be identified and isolated by screening a recombinant combinatorial immunoglobulin library (e.g., an antibody phage display library) with SMRTe to thereby isolate immunoglobulin library members that bind SMRTe. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-SMRTe antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449;, and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-SMRTe antibody (e.g., monoclonal antibody) can be used to isolate SMRTe by standard techniques, such as affinity chromatography or immunoprecipitation. An anti-SMRTe antibody can facilitate the purification of natural SMRTe from cells and of recombinantly produced SMRTe expressed in host cells. Moreover, an anti-SMRTe antibody can be used to detect SMRTe protein (e.g., in a cellular lysate or cell supernatant) in order to evaluate the abundance and pattern of expression of the SMRTe protein. Anti-SMRTe antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a SMRTe protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., SMRTe proteins, mutant forms of SMRTe proteins, fusion proteins, and the like).

The recombinant expression vectors of the invention can be designed for expression of SMRTe proteins in prokaryotic or eukaryotic cells. For example, SMRTe proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in SMRTe activity assays, (e.g., direct assays or competitive assays described in detail below), or to generate antibodies specific for SMRTe proteins, for example. In a preferred embodiment, a SMRTe fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11 d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the SMRTe expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, SMRTe proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g, the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to SMRTe mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The anti sense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a SMRTe protein can be expressed in bacterial cells such as E. coli, insect cells, yeast, or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a SMRTe protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a SMRTe protein. Accordingly, the invention further provides methods for producing a SMRTe protein using the host cells of the invention. In one embodiment, the method comprises culturing the host cell of invention (into which a recombinant expression vector encoding a SMRTe protein has been introduced) in a suitable medium such that a SMRTe protein is produced. In another embodiment, the method further comprises isolating a SMRTe protein from the medium or the host cell.

The host cells of the invention can also be used to produce non-human transgenic animals. For example, in one embodiment, a host cell of the invention is a fertilized oocyte or an embryonic stem cell into which SMRTe-coding sequences have been introduced. Such host cells can then be used to create non-human transgenic animals in which exogenous SMRTe sequences have been introduced into their genome or homologous recombinant animals in which endogenous SMRTe sequences have been altered. Such animals are useful for studying the function and/or activity of a SMRTe and for identifying and/or evaluating modulators of SMRTe activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SMRTe gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a SMRTe-encoding nucleic acid into the male pronuclei of a fertilized oocyte, e.g., by microinjection, retroviral infection, and allowing the oocyte to develop in a pseudopregnant female foster animal. The SMRTe cDNA sequence of SEQ ID NO:1 can be introduced as a transgene into the genome of a non-human animal. Alternatively, a nonhuman homologue of a human SMRTe gene, such as a mouse or rat SMRTe gene, can be used as a transgene. Alternatively, a SMRTe gene homologue, such as another SMRTe family member, can be isolated based on hybridization to the SMRTe cDNA sequences of SEQ ID NO:1, 3, 4, or 6, and used as a transgene. Intronic sequences and polyadenylation signals can also be included in the transgene to increase the efficiency of expression of the transgene. A tissue-specific regulatory sequence(s) can be operably linked to a SMRTe transgene to direct expression of a SMRTe protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a SMRTe transgene in its genome and/or expression of SMRTe mRNA in tissues or cells of the animals. A transgenic founder animal can then be used to breed additional animals carrying the transgene. Moreover, transgenic animals carrying a transgene encoding a SMRTe protein can further be bred to other transgenic animals carrying other transgenes.

To create a homologous recombinant animal, a vector is prepared which contains at least a portion of a SMRTe gene into which a deletion, addition or substitution has been introduced to thereby alter, e.g., functionally disrupt, the SMRTe gene. The SMRTe gene can be a human gene (e.g., the cDNA of SEQ ID NO: 1), but more preferably, is a non-human homologue of a human SMRTe gene such as a murine SMRTe gene (i.e., SEQ ID NO: 4). For example, a mouse SMRTe gene can be used to construct a homologous recombination vector suitable for altering an endogenous SMRTe gene in the mouse genome. In a preferred embodiment, the vector is designed such that, upon homologous recombination, the endogenous SMRTe gene is functionally disrupted (i.e., no longer encodes a functional protein; also referred to as a “knock out” vector). Alternatively, the vector can be designed such that, upon homologous recombination, the endogenous SMRTe gene is mutated or otherwise altered but still encodes functional protein (e.g., the upstream regulatory region can be altered to thereby alter the expression of the endogenous SMRTe protein). In the homologous recombination vector, the altered portion of the SMRTe gene is flanked at its 5′ and 3′ ends by additional nucleic acid sequence of the SMRTe gene to allow for homologous recombination to occur between the exogenous SMRTe gene carried by the vector and an endogenous SMRTe gene in an embryonic stem cell. The additional flanking SMRTe nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced SMRTe gene has homologously recombined with the endogenous SMRTe gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al (1992) Proc. Natl. Acad. Sci USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The SMRTe nucleic acid molecules, fragments of SMRTe proteins, and anti-SMRTe antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a SMRTe protein or an anti-SMRTe antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express SMRTe protein (e.g., via a recombinant expression vector in a host cell in gene therapy applications), to detect SMRTe mRNA (e.g., in a biological sample) or a genetic alteration in an SMRTe gene, and to modulate SMRTe activity, as described further below. The SMRTe proteins can be used to treat disorders characterized by insufficient or excessive production of an SMRTe substrate or production of SMRTe inhibitors. In addition, the SMRTe proteins can be used to screen for naturally occurring SMRTe substrates, to screen for drugs or compounds which modulate SMRTe activity, as well as to treat disorders characterized by insufficient or excessive production of SMRTe protein or production of SMRTe protein forms which have decreased, aberrant or unwanted activity compared to SMRTe wild type protein. Moreover, the anti-SMRTe antibodies of the invention can be used to detect and isolate SMRTe proteins, regulate the bioavailability of SMRTe proteins, and modulate SMRTe activity.

A. Screening Assays:

The invention provides a method (also referred to herein as a “screening assay”) for identifying modulators, i.e., candidate or test compounds or agents (e.g., peptides, peptidomimetics, small molecules, or other drugs) which bind to SMRTe proteins, have a stimulatory or inhibitory effect on, for example, SMRTe expression or SMRTe activity, or have a stimulatory or inhibitory effect on, for example, the interaction of a SMRTe protein with another transcriptional regulator such as a SMRTe family member corepressor, a non-SMRTe corepressor; a TBP associated factor, or a transcription factor, e.g., a nuclear hormone receptor.

In one embodiment, the invention provides assays for screening candidate or test compounds which bind to or modulate the activity of a SMRTe protein or polypeptide or biologically active portion thereof. In another embodiment, the invention provides assays for screening candidate or test compounds which bind to or modulate the activity of a SMRTe target molecule. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection.

Candidate modulators can be purified (or substantially purified) molecules or can be one component of a mixture of compounds (e.g., an extract or supernatant obtained from cells; Ausubel et al., supra). In a mixed compound assay, SMRTe expression or activity, e.g., corepressor activity, is tested against progressively smaller subsets of the candidate compound pool (e.g., produced by standard purification techniques, e.g., HPLC or FPLC) until a single compound or minimal compound mixture is demonstrated to modulate SMRTe expression or activity.

Candidate SMRTe modulators include peptide as well as non-peptide molecules (e.g., peptide or non-peptide molecules found, e.g., in a cell extract, mammalian serum, or growth medium on which mammalian cells have been cultured).

The biological library approach is limited to peptide libraries, while the other approaches are applicable to peptide, non-peptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

Determining the ability of the SMRTe protein to bind to or interact with a SMRTe target molecule can be accomplished by one of numerous methods, for example, by coupling the test compound with a radioisotope or enzymatic label such that binding of the test compound to the SMRTe can be determined by detecting the labeled compound in a complex. For example, test compounds can be labeled with ¹²⁵I, ³⁵S, ¹⁴C, ³²P, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a preferred embodiment, the assay comprises contacting a cell which expresses SMRTe and a SMRTe target molecule, or a biologically- or functionally-active portion of either or both of these molecules, to form an assay mixture, contacting the assay mixture with a test compound, and determining the ability of the test. compound to modulate the interaction between SMRTe and the target molecule, wherein determining the ability of the test compound to modulate the interaction comprises determining the ability of the test compound to preferentially bind to SMRTe as compared to the ability of the test compound to bind to the SMRTe target molecule, or a biologically active portion thereof. As used herein, a “target molecule” is a molecule with which SMRTe protein binds or interacts in nature, for example, a nuclear hormone receptor but may also include, e.g., another SMRTe family member corepressor, a non-SMRTe corepressor, a TBP associated factor, a transcription factor, or any component involved in gene regulation at the level of transcription. In addition, the assay may be a cell-free assay or cell-based assay. In a related embodiment, the assay is performed, wherein determining the ability of the test compound to modulate the interaction between SMRTe and a SMRTe target molecule comprises determining the ability of the test compound to preferentially bind to the SMRTe target molecule, or biologically- or functionally-active portion thereof, as compared to the ability of the test compound to bind to SMRTe. In yet another related embodiment, the foregoing assays are preformed using a target molecule that is a nuclear hormone receptor, and further, tested in the presence and/or absence of receptor ligand, i.e., hormone (e.g., a steroid hormone).

In another embodiment, an assay is a cell-based assay comprising contacting a cell expressing a SMRTe target molecule with a test compound and determining the ability of the test compound to modulate (e.g. stimulate or inhibit) the activity, e.g., corepressor activity of SMRTe on the SMRTe target molecule. Determining the ability of the test compound to modulate the activity of the SMRTe target molecule can be accomplished, for example, by determining the effect of the compound on the ability of SMRTe to bind to or interact with the SMRTe target molecule. Determining the ability of the SMRTe protein to bind to or interact with a SMRTe target molecule can be accomplished by one of the methods described above for determining direct binding. In a preferred embodiment, determining the ability of the SMRTe protein to bind to or interact with a SMRTe target molecule can be accomplished by determining the activity of the target molecule. For example, the activity of the target molecule can be determined by detecting changes in target molecule-mediated transcription (e.g., nuclear receptor-mediated transcription).

In certain embodiments of the above assay methods of the present invention, it may be desirable to immobilize either SMRTe or its target molecule to facilitate separation of complexed from uncomplexed forms of one or both of the proteins, as well as to accommodate automation of the assay. Binding of a test compound to SMRTe, or interaction of SMRTe with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/SMRTe fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or SMRTe protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SMRTe binding or activity determined using standard techniques.

Other techniques for immobilizing proteins on matrices can also be used in the screening assays of the invention. For example, either SMRTe or its target molecule can be immobilized utilizing conjugation of biotin and streptavidin. Biotinylated SMRTe or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SMRTe or target molecules but which do not interfere with binding of the SMRTe protein to its target molecule can be derivatized to the wells of the plate, and unbound target or SMRTe trapped in the wells by antibody conjugation. Methods for detecting such complexes, in addition to those described above for the GST-immobilized complexes, include immunodetection of complexes using antibodies reactive with the SMRTe or target molecule, as well as enzyme-linked assays which rely on detecting an enzymatic activity associated with the SMRTe or target molecule.

In another embodiment, modulators of SMRTe expression are identified in a method wherein a cell is contacted with a candidate compound and the expression of SMRTe mRNA or protein in the cell is determined. The level of expression of SMRTe mRNA or protein in the presence of the candidate compound is compared to the level of expression of SMRTe mRNA or protein in the absence of the candidate compound. The candidate compound can then be identified as a modulator of SMRTe expression based on this comparison. For example, when expression of SMRTe mRNA or protein is greater (statistically significantly greater) in the presence of the candidate compound than in its absence, the candidate compound is identified as a stimulator of SMRTe mRNA or protein expression. Alternatively, when expression of SMRTe mRNA or protein is less (statistically significantly less) in the presence of the candidate compound than in its absence, the candidate compound is identified as an inhibitor of SMRTe mRNA or protein expression. The level of SMRTe mRNA or protein expression in the cells can be determined by methods described herein for detecting SMRTe mRNA or protein.

In yet another aspect of the invention, the SMRTe proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with SMRTe (“SMRTe-binding proteins” or “SMRTe-bp” or “target molecules) and are involved in SMRTe activity as described in the appended example.

The two-hybrid system is based on the modular nature of most transcription factors, which consist of separable DNA-binding and activation domains. Briefly, the assay utilizes two different DNA constructs. In one construct, the gene that codes for a SMRTe protein or a portion of a SMRTe protein, e.g. a receptor interacting domain is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey”, proteins are able to interact, in vivo, forming a SMRTe-dependent complex, the DNA-binding and activation domains of the transcription factor are brought into close proximity. This proximity allows transcription of a reporter gene (e.g., LacZ or β gal) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SMRTe protein. In preferred embodiments a ligand for the nuclear hormone receptor (e.g., a steroid) can be added to the assay to challenge the binding of SMRTe to the nuclear hormone receptor. In these embodiments compounds that inhibit or down modulate the interaction among SMRTe and the receptor can be identified by reduction in reporter gene readout when compared to the reporter gene readout in the absence of compound.

In other preferred embodiments the binding of SMRTe to nuclear hormone receptors can be exploited to discover novel compounds which have a steroid hormone activity. In such embodiments, ligand is omitted from the assay and compounds which decrease the interaction among SMRTe and the receptor can be identified by enhancing the reporter gene readout when compared to the reporter gene readout in the absence of compound.

SMRTe proteins or polypeptides, biologically active portions of SMRTe, SMRTe-derived peptide, as well as fusion proteins thereof, are particularly suited to use in screening assays, for example, for identifying SMRTe corepressor agonists, SMRTe corepressor antagonists (e.g., SMRTe corepressor “dominant negatives”), partial corepressor agonists and/or partial corepressor antagonists. As used herein, the term “partial agonist” or “partial antagonist” includes a molecule or compound which induces a distinct or different conformation of the SMRTe corepressor from that induced via interaction with a SMRTe corepressor agonist or antagonist, respectively. Accordingly, in a preferred embodiment the present invention features a method of identifying a compound which modulates SMRTe corepressor activity or SMRTe target molecule activity, comprising contacting a composition or cell comprising at least a SMRTe target molecule and a SMRTe protein or polypeptide, a biologically active portion of SMRTe, a SMRTe-derived peptide, or a fusion protein thereof, with a test compound, an optionally a hormone or ligand of said SMRTe target molecule, and determining the activity of said SMRTe target molecule such that a compound is identified. The step of determining the activity of such a compound can include determining, for example, transcriptional activity or determining, for example, a conformational change in said SMRTe molecule, or portion thereof, or SMRTe target molecule. Alternatively, The step of determining the activity of such a compound can include any other detecting or determining methodology described herein.

In yet another aspect, the present invention features methods of identifying compounds which modulate SMRTe corepressor activity which involve the use of mutant SMRTe proteins, polypeptides, biologically active portions of SMRTe and/or SMRTe-derived peptides. For example, the present inventors have demonstrated that certain domains of SMRTe, e.g., the SNC domain within SMRTe-derived proteins has the ability to repress transcriptional activity. Accordingly, it is within the scope of the present invention to mutate the SNC domain of the SMRTe proteins, polypeptides, biologically active portions of SMRTe and/or SMRTe-derived peptides and test the protein activity on a target molecule of interest. Mutant SMRTe proteins, polypeptides, biologically active portions of SMRTe and/or SMRTe-derived peptides are also useful in screening for compounds which modulate SMRTe corepressor activity in a manner different from native SMRTe.

This invention further pertains to novel agents identified by the above-described screening assays. A molecule that modulates SMRTe expression or activity is considered useful in the invention; such a molecule can be used, for example, as a therapeutic to modulate cellular levels of SMRTe or to modulate a SMRTe activity.

Furthermore, a molecule that promotes a decrease in SMRTe expression or activity is useful for increasing the efficacy of hormone treatments of disorders involving, for example, a nuclear hormone receptor-mediated disorder.

A molecule that promotes an increase in SMRTe expression or activity is also considered useful in the invention. Such a molecule can be used, for example, as a therapeutic to increase cellular levels of SMRTe or to increase SMRTe binding activity and thereby decrease the activity of certain nuclear hormone receptors. Thus, a molecule that promotes a increase in SMRTe activity is useful in a variety of situations for treating a variety of hormone-induced and hormone-related disorders, e.g., cancer.

Accordingly, it is. within the scope of this invention to further use an agent identified as described herein in an appropriate animal model. For example, an agent identified as described herein (e.g., a SMRTe modulating agent, an antisense SMRTe nucleic acid molecule, a SMRTe-specific antibody, a SMRTe-binding partner or a novel compound which has steroid activity or inhibits a steroid activity) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the SMRTe nucleotide sequences, described herein, can be used to map the location of the SMRTe genes on a chromosome. The mapping of the SMRTe sequences to chromosomes is an important first step in correlating these sequences with genes associated with disease.

Briefly, SMRTe genes can be mapped to chromosomes by preparing PCR primers (preferably 15-25 bp in length) from the SMRTe nucleotide sequences. Computer analysis of the SMRTe sequences can be used to predict primers that do not span more than one exon in the genomic DNA, thus complicating the amplification process. These primers can then be used for PCR screening of somatic cell hybrids containing individual human chromosomes. Only those hybrids containing the human-s gene corresponding to the SMRTe sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the SMRTe nucleotide sequences to design oligonucleotide primers, sublocalization can be achieved with panels of fragments from specific chromosomes. Other mapping strategies which can similarly be used to map a SMRTe sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple. detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the SMRTe gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The SMRTe sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the SMRTe nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The SMRTe nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 or SEQ ID NO:4 can comfortably provide positive individual identification with a panel of perhaps 10 to 1,000 primers which each yield a noncoding amplified sequence of 100 bases. If predicted coding sequences, such as those in SEQ ID NO:3 or SEQ ID NO:6 are used, a more appropriate number of primers for positive individual identification would be 500-2,000.

If a panel of reagents from SMRTe nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial SMRTe Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g. PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1 or SEQ ID NO:4 are particularly appropriate for this use as greater numbers of polymorphisms occur in the noncoding regions, making it easier to differentiate individuals using this technique. Examples of polynucleotide reagents include the SMRTe nucleotide sequences or portions thereof, e.g., fragments derived from the noncoding regions of SEQ ID NO: 1 or SEQ ID NO:4, having a length of at least 20 bases, preferably at least 30 bases.

The SMRTe nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such SMRTe probes can be used to identify tissue by species and/or by organ type.

In a similar fashion, these reagents, e.g., SMRTe primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining SMRTe protein and/or nucleic acid expression as well as SMRTe activity, in the context of a biological sample (e.g., blood, serum, cells, tissue) to thereby determine whether an individual is afflicted with a disease or disorder, or is at risk of developing a disorder, associated with aberrant SMRTe expression or activity. The invention also provides for prognostic (or predictive) assays for determining whether an individual is at risk of developing a disorder associated with SMRTe protein, nucleic acid expression or activity. For example, mutations in a SMRTe gene can be assayed in a biological sample. Such assays can be used for prognostic or predictive purpose to thereby prophylactically treat an individual prior to the onset of a disorder characterized by or associated with SMRTe protein, nucleic acid expression or activity.

Another aspect of the invention pertains to monitoring the influence of agents (e.g., drugs, compounds) on the expression or activity of SMRTe in clinical trials.

These and other agents are described in further detail in the following sections.

1. Diagnostic Assays

An exemplary method for detecting the presence or absence of SMRTe protein or nucleic acid in a biological sample involves obtaining a biological sample from a test subject and contacting the biological sample with a compound or an agent capable of detecting SMRTe protein or nucleic acid (e.g., mRNA, genomic DNA) that encodes SMRTe protein such that the presence of SMRTe protein or nucleic acid is detected in the biological sample. A preferred agent for detecting SMRTe mRNA or genomic DNA is a labeled nucleic acid probe capable of hybridizing to SMRTe mRNA or genomic DNA. The nucleic acid probe can be, for example, a full-length SMRTe nucleic acid, such as the nucleic acid of SEQ ID NO:1, 3, 4, or 6, or a portion thereof, such as an oligonucleotide of at least 15, 30, 50, 100, 250 or 500 nucleotides in length and sufficient to specifically hybridize under stringent conditions to SMRTe mRNA or genomic DNA. Other suitable probes for use in the diagnostic assays of the invention are described herein.

A preferred agent for detecting SMRTe protein is an antibody capable of binding to SMRTe protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SMRTe mRNA, protein, or genomic DNA in a biological sample in vitro as well as in vivo. For example, in vitro techniques for detection of SMRTe mRNA include Northern hybridizations and in situ hybridizations. In vitro techniques for detection of SMRTe protein include enzyme linked immunosorbent assays (ELISAs), Western blots, immunoprecipitations and immunofluorescence. In vitro techniques for detection of SMRTe genomic DNA include Southern hybridizations. Furthermore, in vivo techniques for detection of SMRTe protein include introducing into a subject a labeled anti-SMRTe antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting SMRTe protein, mRNA, or genomic DNA, such that the presence of SMRTe protein, mRNA or genomic DNA is detected in the biological sample, and comparing the presence of SMRTe protein, mRNA or genomic DNA in the control sample with the presence of SMRTe protein, mRNA or genomic DNA in the test sample.

The invention also encompasses kits for detecting the presence of SMRTe in a biological sample. For example, the kit can comprise a labeled compound or agent capable of detecting SMRTe protein or mRNA in a biological sample; means for determining the amount of SMRTe in the sample; and means for comparing the amount of SMRTe in the sample with a standard. The compound or agent can be packaged in a suitable container. The kit can further comprise instructions for using the kit to detect SMRTe protein or nucleic acid.

2. Prognostic Assays

The diagnostic methods described herein can furthermore be utilized to identify subjects having or at risk of developing a disease or disorder associated with aberrant SMRTe expression or activity. For example, the assays described herein, such as the preceding diagnostic assays or the following assays, can be utilized to identify a subject having or at risk of developing a disorder associated with a misregulation in SMRTe protein activity or nucleic acid expression, such as an alteration in gene regulation resulting in, e.g., a cancer, e.g., a leukemia or breast cancer. Alternatively, the prognostic assays can be utilized to identify a subject having or at risk for developing a disorder associated with a misregulation in SMRTe protein activity or nucleic acid expression, such as an alteration in gene regulation resulting in, e.g., a cancer, e.g., a leukemia or breast cancer. Thus, the present invention provides a method for identifying a disease or disorder associated with aberrant SMRTe expression or activity in which a test sample is obtained from a subject and SMRTe protein or nucleic acid (e.g., mRNA or genomic DNA) is detected, wherein the presence of SMRTe protein or nucleic acid is diagnostic for a subject having or at risk of developing a disease or disorder associated with aberrant SMRTe expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SMRTe expression or activity. For example, such methods can be used to determine whether a subject can be effectively treated with an agent for a disorder associated with an alteration in gene regulation resulting in, e.g., a cancer, e.g., a leukemia or breast cancer. Thus, the present invention provides methods for determining whether a subject can be effectively treated with an agent for a disorder associated with aberrant SMRTe expression or activity in which a test sample is obtained and SMRTe protein or nucleic acid expression or activity is detected (e.g, wherein the abundance of SMRTe protein or nucleic acid expression or activity is diagnostic for a subject that can be administered the agent to treat a disorder associated with aberrant SMRTe expression or activity).

The methods of the invention can also be used to detect genetic alterations in a SMRTe gene, thereby determining if a subject with the altered gene is at risk for a disorder characterized by misregulation in SMRTe protein activity or nucleic acid expression, such as an alteration in gene regulation resulting in, e.g., a cancer, e.g., a leukemia or breast cancer. In preferred embodiments, the methods include detecting, in a sample of cells from the subject, the presence or absence of a genetic alteration characterized by at least one of an alteration affecting the integrity of a gene encoding a SMRTe-protein, or the mis-expression of the SMRTe gene. For example, such genetic alterations can be detected by ascertaining the existence of at least one of 1) a deletion of one or more nucleotides from a SMRTe gene; 2) an addition of one or more nucleotides to a SMRTe gene; 3) a substitution of one or more nucleotides of a SMRTe gene, 4) a chromosomal rearrangement of a SMRTe gene; 5) an alteration in the level of a messenger RNA transcript of a SMRTe gene, 6) aberrant modification of a SMRTe gene, such as of the methylation pattern of the genomic DNA, 7) the presence of a non-wild type splicing pattern of a messenger RNA transcript of a SMRTe gene, 8) a non-wild type level of a SMRTe-protein, 9) allelic loss of a SMRTe gene, and 10) inappropriate post-translational modification of a SMRTe-protein. As described herein, here are a large number of assays known in the art which can be used for detecting alterations in a SMRTe gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the SMRTe-gene (see Abravaya et al. (1995) Nucleic Acids Res 0.23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a SMRTe gene under conditions such that hybridization and amplification of the SMRTe-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SMRTe gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in SMRTe can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in SMRTe can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SMRTe gene and detect mutations by comparing the sequence of the sample SMRTe with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the SMRTe gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type SMRTe sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SMRTe cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a SMRTe sequence, e.g., a wild-type SMRTe sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in SMRTe genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control SMRTe nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SMRTe gene.

Furthermore, any cell type or tissue in which SMRTe is expressed may be utilized in the prognostic assays described herein.

3. Monitoring of Effects During Clinical Trials

Monitoring the influence of agents (e.g., drugs) on the expression or activity of a SMRTe protein (e.g., the modulation of membrane excitability or resting potential) can be applied not only in basic drug screening, but also in clinical trials. For example, the effectiveness of an agent determined by a screening assay as described herein to increase SMRTe gene expression, protein levels, or upregulate SMRTe activity, can be monitored in clinical trials of subjects exhibiting decreased SMRTe gene expression, protein levels, or downregulated SMRTe activity. Alternatively, the effectiveness of an agent determined by a screening assay to decrease SMRTe gene expression, protein levels, or downregulate SMRTe activity, can be monitored in clinical trials of subjects exhibiting increased SMRTe gene expression, protein levels, or upregulated SMRTe activity. In such clinical trials, the expression or activity of a SMRTe gene, and preferably, other genes that have been implicated in, for example, a gene regulation or corepressor associated disorder can be used as a “read out” or markers of the phenotype of a particular cell.

For example, and not by way of limitation, genes, including SMRTe, that are modulated in cells by treatment with an agent (e.g., compound, drug or small molecule) which modulates SMRTe activity (e.g., identified in a screening assay as described herein) can be identified. Thus, to study the effect of agents on a gene regulation or corepressor associated disorder, for example, in a clinical trial, cells can be isolated and RNA prepared and analyzed for the levels of expression of SMRTe and other genes implicated in the associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of SMRTe or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a SMRTe protein, mRNA, or genomic DNA in the preadministration sample; (iii) obtaining one or more post-administration samples from the subject; (iv) detecting the level of expression or activity of the SMRTe protein, mRNA, or genomic DNA in the post-administration samples; (v) comparing the level of expression or activity of the SMRTe protein, mRNA, or genomic DNA in the pre-administration sample with the SMRTe protein, mRNA, or genomic DNA in the post administration sample or samples; and (vi) altering the administration of the agent to the subject accordingly. For example, increased administration of the agent may be desirable to increase the expression or activity of SMRTe to higher levels than detected, i.e., to increase the effectiveness of the agent. Alternatively, decreased administration of the agent may be desirable to decrease expression or activity of SMRTe to lower levels than detected, i.e. to decrease the effectiveness of the agent. According to such an embodiment, SMRTe expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant SMRTe expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patients “drug response phenotype”, or “drug response genotype”.) Thus, another aspect of the invention provides methods for tailoring an individual's prophylactic or therapeutic treatment with either the SMRTe molecules of the present invention or SMRTe modulators according to that individual's drug response genotype Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant SMRTe expression or activity, by administering to the subject a SMRTe or an agent which modulates SMRTe expression or at least one SMRTe activity. Subjects at risk for a disease which is caused or contributed to by aberrant SMRTe expression or activity can be identified by, for example, any or a combination of diagnostic or prognostic assays as described herein. Administration of a prophylactic agent can occur prior to the manifestation of symptoms characteristic of the SMRTe aberrancy, such that a disease or disorder is prevented or, alternatively, delayed in its progression. Depending on the type of SMRTe aberrancy, for example, a SMRTe, SMRTe agonist or SMRTe antagonist agent can be used for treating the subject. The appropriate agent can be determined based on screening assays described herein.

2. Therapeutic Methods

Another aspect of the invention pertains to methods of modulating SMRTe expression or activity for therapeutic purposes. Accordingly, in an exemplary embodiment, the modulatory method of the invention involves contacting a cell with a SMRTe or agent that modulates one or more of the activities of SMRTe protein activity associated with the cell. An agent that modulates SMRTe protein activity can be an agent as described herein, such as a nucleic acid or a protein, a naturally-occurring target molecule of a SMRTe protein (e.g., a SMRTe substrate), a SMRTe antibody, a SMRTe agonist or antagonist, a peptidomimetic of a SMRTe agonist or antagonist, or other small molecule. In one embodiment, the agent stimulates one or more SMRTe activities. Examples of such stimulatory agents include active SMRTe protein and a nucleic acid molecule encoding SMRTe that has been introduced into the cell. In another embodiment, the agent inhibits one or more SMRTe activities. Examples of such inhibitory agents include antisense SMRTe nucleic acid molecules, anti-SMRTe antibodies, and SMRTe inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a SMRTe protein or nucleic acid molecule. In one embodiment, the method involves administering an agent (e.g., an agent identified by a screening assay described herein), or combination of agents that modulates (e.g., upregulates or downregulates) SMRTe expression or activity. In another embodiment, the method involves administering a SMRTe protein or nucleic acid molecule as therapy to compensate for reduced or aberrant SMRTe expression or activity.

Stimulation of SMRTe activity is desirable in situations in which SMRTe is abnormally downregulated and/or in which increased SMRTe activity is likely to have a beneficial effect. For example, stimulation of SMRTe activity is desirable in situations in which a SMRTe is downregulated and/or in which increased SMRTe activity is likely to have a beneficial effect. Likewise, inhibition of SMRTe activity is desirable in situations in which SMRTe is abnormally upregulated and/or in which decreased SMRTe activity is likely to have a beneficial effect.

3. Pharmacogenomics

The SMRTe molecules of the present invention, as well as agents, or modulators which have a stimulatory or inhibitory effect on SMRTe activity (e.g., SMRTe gene expression) as identified by a screening assay described herein can be administered to individuals to treat (prophylactically or therapeutically) SMRTe-associated disorders associated with aberrant or unwanted SMRTe activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a SMRTe molecule or SMRTe modulator as well as tailoring the dosage and/or therapeutic regimen of treatment with a SMRTe molecule or SMRTe modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a SMRTe protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a SMRTe molecule or SMRTe modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a SMRTe molecule or SMRTe modulator, such as a modulator identified by one of the exemplary screening assays described herein.

This invention is further illustrated by the following examples which should not be construed as limiting.

### EXEMPLIFICATION

Throughout the examples, the following materials and methods are used unless otherwise stated.

Materials and Methods

Library Screening A 5′-stretched gt11 HeLa cDNA library was screened for human SMRTe according to the manufacturer's protocol (Clontech). Mouse SMRTe was isolated from a ACT mouse embryonic cDNA library. The cDNA inserts were cloned into the pbluescript vector, and the nucleotide sequences were determined using standard techniques and analyzed using the GCG package (University of Wisconsin).

Transient Transfection—Transient transfections were carried out using HeLa cells maintained in DMEM supplemented with 10% FBS. About 12 hr before transfection, 10⁴ cells were seeded into 12-well plates and transiently transfected using a standard calcium phosphate precipitate method (Li et al. (1997) Proc. Natl. Acad. Sci. USA 94, 8479-8484). Cells were then washed, refed, and, 48 hr post-transfection, harvested and processed for luciferase and P-galactosidase assays as described (Li et al. (1997) Proc. Natl. Acad. Sci. USA 94, 8479-8484).

Immunoblot Analysis—SMRTe proteins were detected by immunoblot by first using SDS polyacrylamide gel electrophoresis (PAGE) followed by electroblotting onto nitrocellulose using standard techniques (Harlow, E. & Lane, D. (1988) Antibodies: A Laboratory Manual (Cold Spring Harbor Lab. Press, Plainview, N.Y.). Proteins bound to nitrocellulose were then probed with affinity-purified anti-SMRT rabbit polyclonal antibody (Upstate Biotechnology, Lake Placid, N.Y.) and visualized using a 5-bromo-4-chloro-3-indolyl phosphate/nitroblue tetrazolium color reaction (Vector Laboratories) or the ECL kit (Amersham Pharmacia).

Cell Cycle Assay—The cell cycle assays were performed by synchronizing cells by collecting mitotic cells every 2 hr by mitotic shake-off followed by seeding into tissue culture plates. Cells were harvested by trypsinization and enumerated using a hemocytometer. The cells were then lysed in SDS sample buffer, and cellular proteins were separated by SDS-PAGE and processed for immunoblotting as described above.

Immunocytochemistry—Immunocytochemistry was performed using HeLa and A549 cells grown on coverglasses in 12-well plate for at least 24 hr prior to analysis. Briefly, cells were washed twice with PBS and fixed in methanol/acetone (1:1) for 1 min on dry ice and incubated with affinity-purified anti-SMRT antibody (1:100 dilution). After washing, a fluorescein isothiocyanate-conjugated goat anti-rabbit secondary antibody was added, and the cells were later counterstained with 4′,6-diamidino-2-phenylindole dihydrochloride hydrate (Sigma) as described (Dyck et al. (994) Cell 76, 333-343). Samples were imaged on an epi-fluorescent microscope (Olympus IX-70) with a back-illuminated charge-coupled device camera (Princeton Instruments, Trenton, N.J., 1,000×800) and METAMORPH software (Universal Imaging, Media, Pa.).

In Situ Hybridization—Embryos at different developmental stages were fixed for 2 hr in 4% paraformaldehyde, serially dehydrated, cleared in xylene, and embedded in paraffin. Sections (7 mm) were cut and mounted on ProbeOn Plus slide (Fisher Scientific), deparaffinized, and processed for in situ hybridization using standard techniques (Harland, R. M. (1991) Methods Cell. Biol. 36, 685-695; Henrique et al. (1995) Nature 375, 787-790.

### EXAMPLE 1

### Identification and Characterization of SMRTe cDNAs

In this example, the identification and characterization of the genes encoding human and murine SMRTe are described.

To isolate the cDNA that encodes the human SMRTe 270-kDa protein, a HeLa cDNA library was screened using a DNA probe corresponding to the first transcriptional repression domain between amino acids 137 and 475 of SMRT (Chen et al. (1995) Nature 377, 454-457). Initially, two positive clones were identified that both contain sequences identical to SMRT downstream from the ninth amino acid, but have distinct upstream sequences. Further sequencing analyses revealed that the upstream sequences of both clones contain a continuous ORF, indicating that they are fragments of a longer SMRT isoform. Three further screenings were conducted, resulting in the isolation of 11 overlapping clones that together span an additional 3,190 nucleotides upstream from the ninth amino acid of SMRT. Accordingly, this novel SMRT-related transcript having an novel extended region was termed SMRTe (SMRT-extended) to distinguish it from SMRT previously described (Chen et al. (1995) Nature 377, 454-457). A clone comprising the entire coding region of human SMRTe was deposited with the American Type Culture Collection (ATCC®) Rockville, Md. on ______, and assigned Accession No. ______.

Subsequently, the murine SMRTe cDNA was also isolated by using the foregoing novel human SMRTe as a probe, indicating that the SMRTe isoform is present in both human and mouse. The sequence for human SMRTe and murine SMRTe have been deposited in the GenBank database under, respectively, Accession Nos. AF125672 and AF125671 (see Park et al. (1999) PNAS 95, 3519-1524).

A characterization of these sequences showed that human SMRTe contains 2,507 amino acid residues with a calculated molecular mass of 273,234 Daltons (Da), whereas murine SMRTe contains 2,462 amino acids (see, e.g., FIG. 1). The human and murine SMRTe proteins were determined to share 87% identity, indicating that the SMRTe gene is highly conserved. In addition, a murine clone was identified that lacks a large internal fragment and contains only the N-terminal 609 amino acid region and an unrelated 64 amino acid tail (FIG. 1).

The human SMRTe protein was determined to share 44% identity with human N-CoR (Wang et al. (1998) PNAS 95, 10860-10865), whereas murine SMRTe was determined to share 42% identity with murine N-CoR, indicating that SMRTe and N-CoR are partially related. Interestingly, an N-terminal domain between amino acid residues 166 and 429 is strikingly conserved between SMRTe and N-CoR (86% identity and 91% similarity) (FIGS. 3 and 4). Accordingly, this domain was termed the SMRTe and N-CoR conserved (SNC) domain. The SNC domain was determined to have at the N terminus an amphipathic-helix containing five hydrophobic heptad repeats is present (FIG. 3).

The SNC domain is followed by two conserved repeats known as the SANT (SWI3, ADA2, N-CoR, and TFIIIB B″) domains (Aasland et al. (1996) Trends Biochem. Sci. 21, 87-88). The two SANT motifs are only marginally related to one another within the same protein (30% identity), whereas the individual motif is highly conserved between SMRTe and N-CoR in both the human and mouse (>75% identity) (FIG. 4). Therefore, the N-terminal SANT motif is referred to as SANT-A and the C-terminal motif as SANT-B (FIGS. 1 and 4). The SANT-A and SANT-B motifs are separated by an intervening sequence of approximately 120 amino acids, which contains a polyglutarnine track and a charged acidic-basic region followed by a short segment that also is highly conserved between SMRTe and N-CoR (FIG. 1).

In addition, a number of additional motifs were determined to be present in SMRTe such as an acidic-basic domain, SIT repeated motifs, KGH repeated motifs, an serine/glycine-rich region; SMRTe repression domains (SRD), and nuclear receptor interacting domains (RID) (see, e.g., FIG. 3 and Li et al. (1997) Mol. Endocrinol. 11, 2025-2037).

Based on the foregoing it was concluded that a full-length isoform of SMRT termed SMRTe has been identified. In addition, identification of the N-terminal extended domain of SMRTe reveals several interesting relationships with N-CoR. First, that this region contains a 300 amino acid domain that shares more than 90% similarity with N-CoR. Because this region of N-CoR is involved in both transcriptional repression and protein-protein interactions, the high homology indicates that this domain of SMRTe has similar function. Accordingly, it was determined that the highly conserved SNC domain is crucial for transcriptional repression (see, e.g., Example 3). Second, SMRTe contains a unique polyglutamine track that is absent in N-CoR. Polyglutamine tracks are found in a number of transcriptional regulators, and the expansion of glutamines relates to several human diseases (Fischbeck et al. (1997) J. Inherit. Metab. Dis. 20, 152-158; Reddy et al. (1997) Curr. Opin. Cell. Biol. 9, 364-372; and Davies et al. (1998) Lancet 351, 131-133). The unique polyglutamine track in SMRTe indicates that a differential functional property between SMRTe and N-CoR may exist. Third, the two SANT motifs previously found in N-CoR and other transcriptional regulators also are present in SMRTe, indicating that SMRTe is a SANT-containing protein (Aasland et al., (1996) Trends Biochem. Sci. 21, 87-88). It is of note that the SANT motifs in SMRTe and N-CoR are akin to similar motifs found in Myb oncoproteins that mediate DNA binding by resembling homeodomain-like, helix-turn-helix motifs (Frampton et al. (1991) Protein Eng. 4, 891-901; Ogata et al. (1994) Cell 79, 639-648). Thus, the two SANT repeats in SMRTe and N-CoR can contribute to DNA binding as either sequence-specific transcription repressors or by contributing to DNA binding while associating with DNA binding proteins.

In addition or alternatively, the SANT domains can play a role in protein-protein interaction required for assembly of nuclear corepressor complexes. Indeed, the SMRTe SANT-A and SANT-B domains are separated by a polyglutamine track, a highly charged motif, and a conserved segment and these intervening sequences can regulate a functional interaction between the SANT-A and SANT-B motifs.

Finally, it is of note that the N-terminal 160 amino acids of N-CoR interact with mSiah2, which targets N-CoR for proteosome-mediated degradation in a cell-dependent manner (Zhang et al. (1998) Genes Dev. 12, 1775-1780). Importantly, this region of N-CoR is not conserved within SMRTe, indicating that SMRTe may not interact with mSiah2 and that the mechanism of SMRTe turnover may differ from that of N-CoR. In contrast, a component of the HDAC-containing corepressor complex, SAP30, interacts with the N-terminal 312 amino acid of N-CoR (Laherty et al. (1998) Mol. Cell 2, 33-42). This region contains a significant portion of the highly conserved domain, suggesting that SAP30 can interact with SMRTe. Furthermore, amino acids 254-312 of N-CoR have been shown to interact with both Pit1 and mSin3A/B (Xu et al. (1998) Nature 395, 301-306; Heinzel et al. (1997) Nature 387, 43-48). Within this 59 amino acid region, only five residues differ between SMRTe and N-CoR, indicating that this region of SMRTe can interact with Pit1 and mSin3.

Thus, while several isoforms of SMRT and N-CoR have been reported, including, e.g., the SMRT dominant negative form TRAC1, which contains only the C-terminal nuclear receptor-interacting domain, and the N-CoR/RIP 13 form that is similar in size and structure to SMRT, the present invention provides SMRTe, which contains an additional N-terminal domain when compared with the previously identified SMRT (Sande et al. (1996) Mol. Endocrinol. 10, 813-825; Seol et al. (1995) Mol. Endocrinol. 9, 72-85; and Chen et al. (1995) Nature 377, 454-457). Surprisingly, the N-terminal extended sequence of SMRTe exhibits striking similarity with the N-terminal 1,000 amino acid residues of N-CoR, indicating that SMRTe and N-CoR share more related structure and function.

### EXAMPLE 2

### Methods for Identifying Endogenous SMRTe Proteins in Mammalian Cells

In this example, the identification of endogenous SMRTe proteins in mammalian cells, is described.

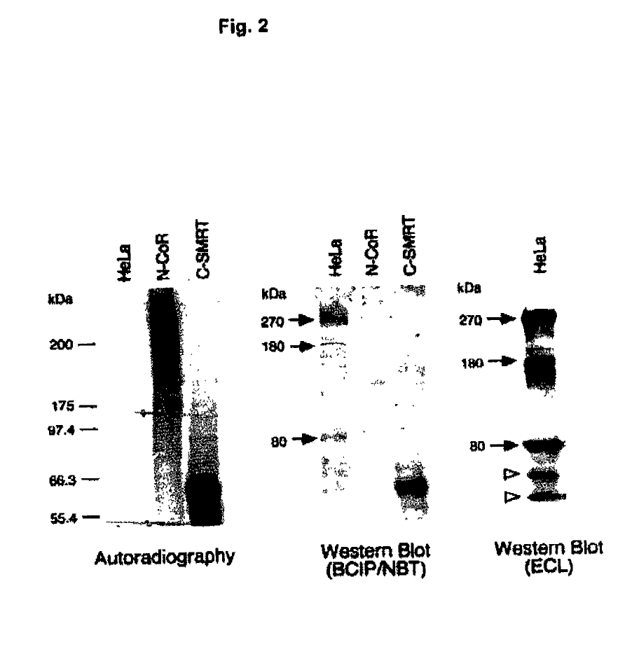

In order to demonstrate the presence of endogenous SMRTe proteins in mammalian cells, an immunoblot was performed using an affinity-purified anti-SMRT antibody to detect the presence of natural SMRT proteins and related SMRTe proteins in a cell extract. HeLa cell nuclear extract, together with positive controls consisting of in vitro-translated N-CoR (6) and C-SMRT (5), were separated by SDS/PAGE. The N-CoR protein migrates as a 270-kDa polypeptide and the C-SMRT as a 60-kDa protein as detected by autoradiography (FIG. 2, Left Panel). By immunoblot, the anti-SMRT antibody reacts strongly with C-SMRT and does not crossreact with N-CoR (FIG. 2, Center Panel). Using the HeLa nuclear extract, the anti-SMRT antibody detects a major polypeptide of 270 kDa that migrates at a position similar to that of N-CoR and recognizes two weak polypeptides of approximately 180 and 80 kDa (FIG. 2, Center Panel). The 180- and 80-kDa bands were more evident when the immunoblot was developed with the ECL+reagents (FIG. 3, Right Panel). Preincubating the antibody with purified SMRT antigen eliminates all three SMRT signals except nonspecific bands. In contrast, preincubating with purified N-CoR antigen does not reduce the SMRT signals. In addition, the same 270-kDa SMRTe protein was also detected in many different cell lines, including CV-1, 293, NB4, MCF7, T47D, and HBL100.

These results indicate that SMRTe is expressed primarily as a 270-kDa protein, in addition to two shorter proteins.

### EXAMPLE 3

### Functional Characterization of SMRTe Activity

In this example, a functional characterization of the SMRTe protein is described.

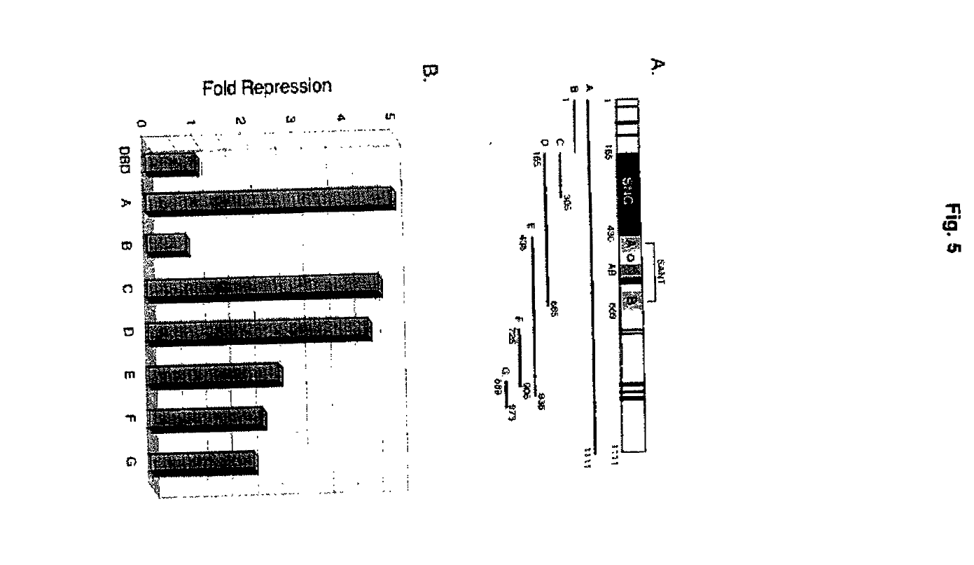

To demonstrate the transcriptional repression function of the N-terminal sequence of SMRTe, the ability of the protein to repress basal transcription of a reporter gene was assayed in mammalian cells. When linked with a Gal4 DNA binding domain (DBD), SMRTe (1-1111) efficiently represses basal transcription from a luciferase reporter containing four copies of Gal4 binding sites (FIGS. 5A and B). To further characterizes this activity, the N-terminal sequence of SMRTe was then divided into overlapping fragments (FIG. 5A) which were individually linked to Gal4 DBD and assayed for their transcriptional repression activities. The results indicate that the N-terminal 140 amino acids of the SNC domain contains strong transcriptional repression activity (FIG. 5B), indicating that at least one role for this SNC domain is to repress basal transcription. In addition, it was observed that regions outside of the SNC domain, except for the N-terminal 165 amino acids, also exhibit some repression activity (FIGS. 5A and B).

Accordingly, it was concluded that, like N-CoR, the N-terminal domain of SMRTe is involved in transcription repression and that the SNC domain is crucial for this function.

### EXAMPLE 4

### Characterization of SMRTe Expression During the Cell Cycle

In this example, a characterization of cell cycle dependent SMRTe expression is described.

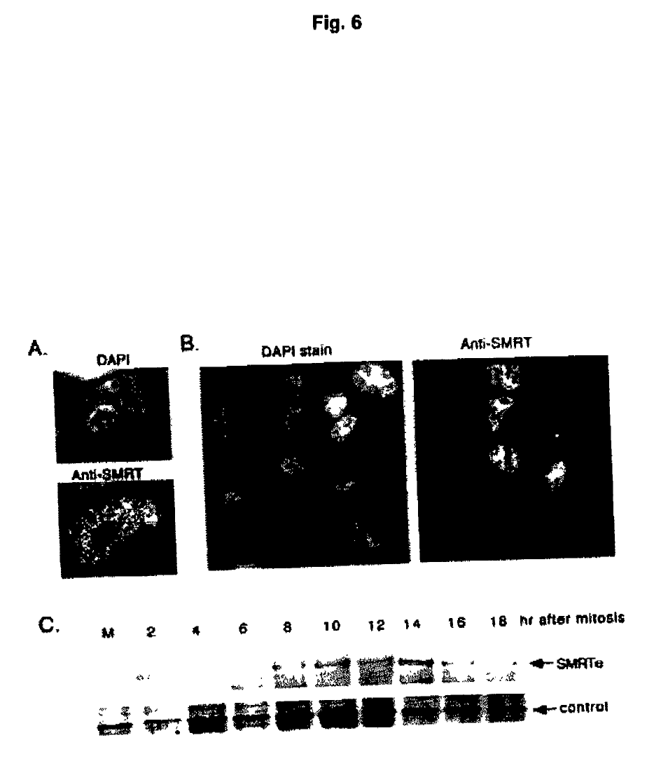

Specifically, by using an affinity purified anti-SMRT antibody, the subcellular distribution of endogenous SMRTe protein was determined using immunofluorescence staining (see FIG. 6). In particular, fine granules were observed in HeLa cell nuclei that are excluded from nucleoli (FIG. 6A). This finding is in contrast with the distribution of overexpressed SMRT (Lin et al. (1998) Nature 391, 811-814). As A549 cells fail to express any detectable SMRTe message by Northern blotting, these cells were used as a negative control in the immunofluorescence study. The overall intensity of SMRT staining in A549 cells is weaker than in the HeLa cells (FIG. 6B, Right Panel). However, a subset of A549 cells was observed that expressed relatively higher levels of SMRTe (FIG. 6, Right Panel). Indeed, it was estimated that approximately 20% of the A549 cells display clearly detectable levels of SMRTe using this assay.

To determine if the fluctuation in immunostaining suggests that SMRTe expression may be regulated in a cell cycle-dependent manner, A549 cells were synchronized and endogenous SMRTe protein levels were analyzed at different time points after release from mitosis using immunoblotting. It was determined that the 270-kDa SMRTe protein level increased at a time when cells normally would enter S phase between 8 and 14 hr after mitosis (see FIG. 6C, Upper Panel). A nonspecific band shows approximately equal intensity in all samples that have been preadjusted by cell number (FIG. 6C, Lower Panel).

Accordingly, these results indicate that SMRTe expression is cell cycle regulated, indicating that SMRTe can play a role in cell cycle progression. For instance, the corepressor can repress expression of cell cycle-specific genes, and thus contribute to regulation of cell cycle progression. It has been observed, for example, that cell cycle-dependent modification of the coactivator CBP occurs (Ait-Si-Ali et al. (1998) Nature 396, 184-186). Alternatively, the corepressor can be involved in other cellular processes occurring at specific stages of the cell cycle, such as DNA replication. For example, SMRTe and N-CoR may function together.

### EXAMPLE 5

### Characterization of SMRTe Tissue Expression in a Mammal

In this example, the characterization of SMRTe expression in a whole embryo is described.

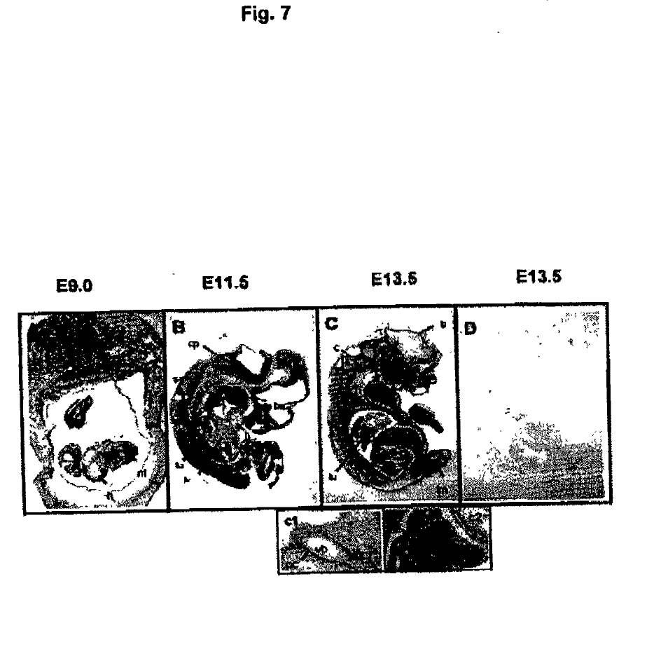

Previously, SMRT message has been detected in all stages of mouse embryos by Northern blotting (Chen et al. (1996) PNAS 93, 7567-7571). To provide further insight into the expression of SMRTe during embryogenesis, the distribution of SMRTe transcripts in early mouse embryos were analyzed by in situ hybridization. Using a digoxigenin (DIG)-labeled antisense mouse SMRTe riboprobe, SMRTe transcripts were detected in thin sections of mouse embryos at embryonic day (E) 9.0, E11.5, and E13.5 postconception (FIG. 7). Typically, SMRTe transcripts are found at E9.0-E13.5 in nearly all tissues with low levels of expression in the heart and liver. The expression in the frontal section of E9.0 is most prominent in the neural tube and undetectable in the heart. In the sagittal section of E11.5, the SMRTe transcripts are high in the condensation of sclerotome, lung, the first bronchial arch, and cerebellar plate (metencephalon). SMRTe levels, however, are low in the liver and the atrium and ventricle of the heart. In the sagittal section of an E13.5 embryo, the SMRTe transcripts are expressed in the lung, brain, and the perichondrium of the head, neck, and the ribs. Little or no expression was observed in the developed vertebrate body, liver, or heart.

These results indicate that SMRTe transcripts are widely expressed in early mouse embryos, supporting a role for SMRTe in multiple biological processes during embryogenesis.

### EXAMPLE 6

### Assay for Screening Modulators of SMRTe Regulation of Gene Transcription

In this example, an assay for measuring SMRTe-mediated gene regulation an identifying modulators thereof, is presented.

It has been observed that SMRTe can affect the expression of genes regulated by, e.g., a nuclear receptor such as TR or RAR. For example, SMRTe can function as a corepressor of the foregoing transcriptional regulators thereby altering or, e.g., decreasing, gene expression controlled by the transcriptional regulator. In addition, based on the functional characterization of the SMRTe in Example 3, it was discovered that the SMRTe is capable of repressing gene transcription. Accordingly, SMRTe can be used as, e.g., a dominant negative regulator of, e.g., undesired gene expression. Moreover, this may be facilitated and/or made promoter specific or regulator specific by fusing to the SMRTe protein, or derivative thereof such as the transcriptional repressor portion of the SMRTe protein, a heterologous DNA-binding or protein-binding protein. Still further, this fusion protein, wild type SMRTe, or a derivative thereof can be assayed for its ability to regulate the promoter of an important gene, e.g., a cell cycle regulated gene, including any art recognized cell cycle regulated gene and/or a gene involved in a cell growth phenotype (including, e.g., a transformed phenotype, such as a leukemia).

Accordingly, eukaryotic cells (e.g., mammalian HeLa cells) can be co-transfected with a reporter construct (encoding, e.g., luciferase) and a plasmid encoding a SMRTe corepressor and optionally a transcriptional regulator. Ideally, the reporter gene is selected for high expression in the absence of SMRTe corepressor activity. Following transfection, cells are harvested, and reporter gene activity as a function of luciferase activity in the presence or absence of a SMRTe repressor molecule is determined as described in the materials and methods subsection above.

In order to determine if SMRTe can affect the gene transcription of other promoters, other gene promoters (including, e.g., viral promoters) may be engineered upstream of the reporter gene and tested as described above. To verify that the cells are transfected with equivalent amounts of constructs encoding SMRTe, immunoblot analysis of SMRTe polypeptide levels using, e.g., an anti-SMRTe polyclonal antisera can be performed.

In addition to determining if SMRTe expression can repress gene transcription, the assay may also employed to test the ability of a compound to enhance or inhibit SMRTe-mediated repression of gene expression.

Accordingly, it will be appreciated that the assay has wide utility in screening modulators of SMRTe-mediated gene regulation. For example, the reporter disclosed herein (see also Example 3) may be used because of the unambiguous signal that can be assayed and because an inhibitor of SMRTe-mediated repression will rescue signal output, i.e., reporter gene expression. Because the amount of SMRTe repression of this promoter is strong, even weak or partial inhibitors of SMRTe activity can be readily assayed.

Moreover, the assay provides a control that can accurately identify compounds that are false positives (e.g., compounds that rescue the signal but also increase the signal in the test reaction) or false negatives (e.g., compounds that produce no signal but also lower the control signal, e.g., cytotoxic compounds) and this insures that inappropriate compounds are not further investigated and that candidate compounds are not erroneously dismissed.

It will be further appreciated that any art recognized compound or library of compounds containing, e.g., a test compound that is protein based, carbohydrate based, lipid based, nucleic acid based, natural organic based, synthetically derived organic based, or antibody based may be screened as a candidate compound that affects SMRTe-mediated regulation of a promoter (i.e., gene expression). Accordingly, any of a number of art recognized high throughput assay techniques may be used in conducting the assay.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 60% homologous to the nucleotide sequence of SEQ ID NO:1, SEQ ID NO:3, SEQ ID NO:4, SEQ ID NO:6, or a complement thereof,
b) a nucleic acid molecule comprising a fragment of at least 949 nucleotides of a nucleic acid comprising the nucleotide sequence of SEQ ID NO:1, SEQ ID NO:3, SEQ ID NO:4, SEQ ID NO:6, or a complement thereof;
c) a nucleic acid molecule which encodes a polypeptide comprising an amino acid sequence at least about 50% homologous to the amino acid sequence of SEQ ID NO:2 or SEQ ID NO:5;
d) a nucleic acid molecule which encodes a fragment of a polypeptide comprising the amino acid sequence of SEQ ID NO:2 or SEQ ID NO:5, wherein the fragment comprises at least 15 contiguous amino acid residues of the amino acid sequence of SEQ ID NO:2 or SEQ ID NO:5; and
e) a nucleic acid molecule which encodes a naturally occurring allelic variant of a polypeptide comprising the amino acid sequence of SEQ ID NO:2 or SEQ ID NO:5, wherein the nucleic acid molecule hybridizes to a complement of a nucleic acid molecule comprising SEQ ID NO: 1, SEQ ID NO:3, SEQ ID NO:4, or SEQ ID NO:6, under stringent conditions.

2. The isolated nucleic acid molecule of claim 1 which is selected from the group consisting of:
a) a nucleic acid molecule comprising the nucleotide sequence of SEQ ID NO:1, SEQ ID NO:3, SEQ ID NO:4, SEQ ID NO:6, or a complement thereof; and
b) a nucleic acid molecule which encodes a polypeptide comprising the amino acid sequence of SEQ ID NO:2 or SEQ ID NO:5.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2 or SEQ ID NO:5, wherein the fragment comprises at least 15 contiguous amino acids of SEQ ID NO:2 or SEQ ID NO:5;
b) a naturally occurring allelic variant of a polypeptide comprising the amino acid sequence of SEQ ID NO:2 or SEQ ID NO:5, wherein the polypeptide is encoded by a nucleic acid molecule which hybridizes to a complement of a nucleic acid molecule comprising SEQ ID NO:1, SEQ ID NO:3, SEQ ID NO:4, SEQ ID NO:6, under stringent conditions; and
c) a polypeptide which is encoded by a nucleic acid molecule comprising a nucleotide sequence which is at least 50% homologous to a nucleic acid comprising the nucleotide sequence of SEQ ID NO: 1, SEQ ID NO:3, SEQ ID NO:4, or SEQ ID NO:6.
d) a polypeptide comprising an amino acid sequence which is at least 30% homologous to the amino acid sequence of SEQ ID NO:2 or SEQ ID NO:5.

9. The isolated polypeptide of claim 8 comprising the amino acid sequence of SEQ ID NO:2 or SEQ ID NO:5.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO: 2 or SEQ ID NO:5;
b) a fragment of a polypeptide comprising the amino acid sequence of SEQ ID NO:2 or SEQ ID NO:5; wherein the fragment comprises at least 15 contiguous amino acids of SEQ ID NO:2 or SEQ ID NO:5; and
c) a naturally occurring allelic variant of a polypeptide comprising the amino acid sequence of SEQ ID NO:2 or SEQ ID NO:5, wherein the polypeptide is encoded by a nucleic acid molecule which hybridizes to a complement of a nucleic acid molecule comprising SEQ ID NO: 1, SEQ ID NO:3, SEQ ID NO:4, SEQ ID NO:6, under stringent conditions;
comprising culturing the host cell of claim 5 under conditions in which the nucleic acid molecule is expressed.

13. A method for detecting the presence of a polypeptide of claim 8 in a sample comprising:
a) contacting the sample with a compound which selectively binds to the polypeptide; and
b) determining whether the compound binds to the polypeptide in the sample to thereby detect the presence of a polypeptide of claim 8 in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule in claim 1 in a sample comprising:
a) contacting the sample with a nucleic acid probe or primer which selectively hybridizes to a complement of the nucleic acid molecule; and
b) determining whether the nucleic acid probe or primer binds to a nucleic acid molecule in the sample to thereby detect the presence of a nucleic acid molecule of claim 1 in the sample.

17. The method of claim 16, wherein the sample comprises mRNA molecules and is contacted with a nucleic acid probe.

18. A kit comprising a compound which selectively hybridizes to a complement of a nucleic acid molecule of claim 1 and instructions for use.

19. A method for identifying a compound which binds to a polypeptide of claim 8 comprising:
a) contacting the polypeptide, or a cell expressing the polypeptide with a test compound; and
b) determining whether the polypeptide binds to the test compound.

20. The method of claim 19, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for SMRTe activity.

21. A method for modulating the activity of a polypeptide of claim 8 comprising contacting the polypeptide or a cell expressing the polypeptide with a compound which binds to the polypeptide in a sufficient concentration to modulate the activity of the polypeptide.

22. A method for identifying a compound which modulates the activity of a polypeptide of claim 8 comprising:
a) contacting a polypeptide of claim 8 with a test compound; and
b) determining the effect of the test compound on the activity of the polypeptide to thereby identify a compound which modulates the activity of the polypeptide.

23. A method for identifying a compound which modulates the activity of a polypeptide of claim 8 comprising:
a) contacting a cell containing a polypeptide of claim 8 with a test compound; and
b) determining the effect of the test compound on the activity of the polypeptide to interact with a SMRTe target molecule thereby identifying a compound which modulates the activity of the polypeptide.

24. The method of claim 23, wherein said activity is corepression of gene regulation at the level of transcription.

25. The method of claim 23, wherein said SMRTe target molecule is a nuclear hormone receptor.

26. The method of claim 25, wherein said contacting is in the presence of a ligand that binds a nuclear hormone receptor.

27. A method for treating or preventing a condition associated with aberrant SMRTe protein or nucleic acid expression or activity comprising,
administering to a subject a therapeutically effective amount of an agent sufficient to modulate said aberrant SMRTe protein or nucleic acid expression or activity in said subject.

28. The method of claim 27, wherein said condition is a cancer.

29. A method for modulating SMRTe-mediated gene regulation in a subject in need thereof comprising,
administering a therapeutically-effective amount of an agent to the subject such that modulation occurs.
